(19) United States
(12) Patent Application Publication
(10) Pub. No.: US 2026/0241104 A1
(43) Pub. Date: Aug. 20, 2026

(54) SYSTEMS AND METHODS FOR DELIVERING A COMPOSITION

(71) Applicant: CONTRALINE, INC., Charlottesville, VA (US)

(72) Inventors: Timothy James BARRY, Charlottesville, VA (US); Kevin S. EISENFRATS, Charlottesville, VA (US); Gregory GROVER, Houston, TX (US); David S. HAZLITT, Lafayette, CO (US); Alissa K. WONG, Boulder, CO (US); Daniel M. KIFLE, Minneapolis, MN (US)

(73) Assignee: CONTRALINE, INC., Charlottesville, VA (US)

(21) Appl. No.: 18/874,438

(22) PCT Filed: Jun. 23, 2023
(86) PCT No.: PCT/US2023/026080

§ 371 Date: Dec. 12, 2024

Related U.S. Application Data
(60) Provisional application No. 63/355,867, filed on Jun. 27, 2022.

(51) Int. Cl. A61M 5/20; A61M 5/145; A61M 5/315

(52) U.S. Cl. CPC A61M 5/2066; A61M 5/1456; A61M 5/31578; A61M 5/31596; A61M 2202/0007; A61M 2205/0216; A61M 2205/121; A61M 2205/3327; A61M 2205/584

(57) ABSTRACT

Delivery systems and methods for delivering a composition are described herein. In particular, apparatus and methods for performing controlled delivery of the composition via a delivery system that includes a delivery device and a cartridge assembly are described. The interface between the delivery device and the cartridge assembly maintains a supported container in a fixed longitudinal position relative to the delivery device and the composition is precisely dispensed from the container in response to an actuation of the delivery device.

### CROSS-REFERENCE TO RELATED APPLICATIONS

This application claims priority to and the benefit of U.S. Provisional Patent Application No. 63/355,867, entitled “Systems and Methods for Delivering a Composition,” filed Jun. 27, 2022, the disclosure of which is incorporated by reference in its entirety.

### BACKGROUND

The embodiments described herein relate generally to delivery devices and methods of delivering a composition, such as biomaterials.

In many fields, it is desirable to inject, implant, or deposit precise amounts of a composition at a target location. For example, in certain applications, the composition may be a biomaterial that is implanted into a small area such as the lumen of a vessel or duct of a patient. Biomaterials are natural or synthetic materials (such as polymers) that are suitable for introduction into living tissues as a therapeutic (to treat, augment, repair, modify, or replace a tissue function of the body) or as a diagnostic. Biomaterials such as hydrogel implants have been shown to be useful for embolization, drug delivery, sealing, filling, and occlusion purposes. Hydrogels are highly hydrated polymer chains or networks that are able to absorb significant volumes of water and can have tunable mechanical properties. In some applications, the biomaterial will form an implant that acts as an occlusion or embolization of a lumen. The occlusion can be used for providing contraception to a subject by occluding the vas deferens, fallopian tube(s), or uterus. Such occlusions can also be used to occlude any other body part, such as ducts, tissues, interstitial spaces, or organs, such as for drug delivery, spacing, sealing, embolizing, or bulking purposes. Known delivery systems, however, do not provide the desired accuracy, precision, and/or repeatability, particularly when micro-volumes are involved. For example, some known delivery systems and methods are specifically designed to produce a spray (e.g., for wound healing, etc.). Because precise control over the total amount delivered, timing of the delivery, rate of delivery and/or delivery force is not often a significant concern for such applications, such known systems are not suitable for applications where delivery of a small, precise amount to a specific location is desired.

Some known systems include a manual delivery device, such as a syringe that is operated by hand to deliver the components. Such manual systems, however, can often result in high variability. For example, the manual force applied by a physician on a hand delivery device can vary from procedure-to-procedure, and those variations are further magnified when considering different physicians performing the delivery procedure on different patients under different operating conditions. The variations in manual force applied on known hand delivery devices may drastically impact the total delivery amount administered to a patient and/or to a specific target area, particularly when the desired amount of compositions is very small (e.g., 0.001 mL to 1 mL). Insufficient delivery of compositions may result in the target area not being properly occluded, embolized, or sealed, which may result in a failed operation. On the other hand, excess delivery of compositions may lead to undesired occlusion, embolization, or sealing of non-target areas, and may be problematic if the non-target areas are sensitive to the compositions or need to remain free from occlusion, embolization, or sealing. Excess delivery of composition may also result in delivery of a final amount greater than the capacity of the target area, resulting in damage. For example, if the volume of the compositions delivered exceeds a capacity of a target vessel, the target vessel may rupture or the tissue may experience a histological response.

To achieve the required precision, some known systems employ a delivery device that produces a force to control the delivery of the composition. Often, the delivery device is a reusable device that is repeatedly reused for the delivery of the composition (e.g., in multiple different applications or procedures). As such, doses of the composition are often maintained in a separate container that is joined to the delivery device and delivered therefrom. However, deviations in the interface between the container and the delivery device may affect the amount of the composition that is delivered. For example, deviations in the positioning of the container relative to the delivery device may result in an imprecise amount of the composition being delivered. In other words, a deviation of the container from a nominal position may result in an excessive or insufficient amount of the composition being delivered.

In some applications, the delivery device is employed in an environment where contaminants are potentially present, such as during a precision manufacturing environment or during a medical procedure. Thus, for the delivery device to be reusable, it may be desirable to limit the contamination of the delivery device. Thus, in such applications the delivery device is often cleaned and/or sterilized following the delivery of the composition. Accordingly, it may be desirable to mitigate potential drawbacks that may result from the cleaning/sterilization process, such as damage to the delivery device or excessive time for cleaning.

Thus, a need exists for devices and methods for delivering precise amounts of a composition. More specifically, a need exists for devices and methods that allow for a container of composition to be repeatedly coupled to and decoupled from a delivery device at a precise location relative to the delivery device. A need also exists for devices and methods that support the reusability of the delivery device by limiting contamination of the delivery device and mitigating potential damage to the delivery device resulting from cleaning and/or sterilization.

### SUMMARY

Delivery devices for delivering compositions are described herein. In particular, apparatuses and methods for performing controlled delivery a composition, such as a biomaterial, are described. More specifically, the composition is delivered from a disposable cartridge assembly via a reusable delivery device. The cartridge assembly and the delivery device can, for example, be employed to form and deliver biomaterials into or onto a body part, such as a body lumen.

In some embodiments, a cartridge assembly includes a container for retaining a composition, such as a biomaterial or a component thereof. The container includes a container body and an elastomeric member disposed within the container body to retain the composition within the container. The cartridge assembly also includes a cartridge chassis having a plurality coupling members distributed about a perimeter of the cartridge chassis. Each of the coupling members is positioned to be received by a coupling interface of a delivery device. The cartridge chassis includes a container clip at least partially surrounding the container body to secure the container in a fixed position relative to the cartridge chassis. A first coupling member of the plurality of coupling members includes a longitudinal positioning feature positioned to engage a first corresponding feature of the coupling interface of the delivery device to prevent longitudinal movement of the cartridge chassis relative to the coupling interface.

In some embodiments, an apparatus includes a housing that defines a coupling interface. The coupling interface is configured to receive a set of coupling members of a cartridge chassis that supports a container. The container includes a container body and an elastomeric member disposed within the container body to retain a composition within the container. The coupling interface includes at least one longitudinal positioning feature positioned to engage a corresponding feature of the cartridge chassis and establish a longitudinal position of the cartridge chassis. Accordingly, the receipt of the coupling members by the coupling interface establishes the container in a fixed position relative to the housing. The apparatus also includes a motive assembly configured to be operatively coupled to the container. The motive assembly is configured to move the elastomeric member within a container body of the container to convey a portion of the composition.

In some embodiments, an apparatus includes a housing that defines a coupling interface. The coupling interface is configured to receive a cartridge chassis that supports a container. The container includes a container body, a plunger, and an elastomeric member disposed within the container body to retain a composition within the container. The coupling interface includes at least one longitudinal positioning feature positioned to engage a corresponding feature of the cartridge chassis and establish a longitudinal position of the cartridge chassis. A motive assembly is positioned within the housing. A drive member is removably coupled to the motive assembly. The drive member includes a first drive member portion and a second drive member portion. A first distal end of the first drive member portion is within the housing. A second distal end of the second drive member portion is configured to be operatively coupled to the container to move the elastomeric member within a container body of the container to convey a portion of the composition in response to actuation of the motive assembly.

The description below and the accompanying figures will provide greater details on the various systems, methods and devices for delivering a composition.

### BRIEF DESCRIPTION OF THE DRAWINGS

FIG. 1 is a schematic illustration of a portion of a delivery system according to an embodiment.

FIG. 2 is a perspective view of a delivery system according to an embodiment.

FIG. 3 is a partially exploded view of the delivery system of FIG. 2.

FIG. 4 is a side view of the delivery system of FIG. 2.

FIG. 5 is a proximal end view of the delivery system of FIG. 2.

FIG. 6 is an overhead view of the delivery system FIG. 2.

FIG. 7 is an exploded view of a container and a connector according to an embodiment and that are coupled to the delivery device shown in FIG. 2.

FIG. 8 is an overhead view of a cartridge chassis of the delivery system of FIG. 2 coupled to the delivery device of the delivery system of FIG. 2.

FIG. 9 is a partial side cross-sectional view of the cartridge chassis and the delivery device taken at line A-A in FIG. 8.

FIG. 10 is a partial side cross-sectional view of the delivery device with the cartridge chassis removed for clarity taken at line A-A in FIG. 8.

FIG. 11 is a perspective view of the partial cross-sectional view of the delivery device depicted in FIG. 10.

FIG. 12 is a partial cross-sectional view of the cartridge chassis and the delivery device taken at line B-B in FIG. 8.

FIG. 13 is a perspective view of the cartridge chassis shown in FIG. 8.

FIG. 14 is a perspective view of the cartridge chassis of FIG. 13.

FIG. 15 is an overhead view of the cartridge chassis of FIG. 13.

FIG. 16 is a side view of the cartridge chassis of FIG. 13.

FIG. 17 is a partial side cross-sectional view of the cartridge chassis and a container taken at line C-C in FIG. 6.

FIG. 18 is a side view of the delivery device shown in FIG. 2 showing certain internal components in broken lines.

FIG. 19 is a partially exploded view of the delivery device of FIG. 18 according to an embodiment.

### DETAILED DESCRIPTION

The term “about” when used in connection with a referenced numeric indication means the referenced numeric indication plus or minus up to 10% of that referenced numeric indication. For example, “about 100” means from 90 to 110.

The term “substantially” when used in connection with, for example, a geometric relationship, a numerical value, and/or a range is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. By way of another example, a structure defining a volume that is “substantially 0.50 milliliters (mL)” is intended to convey that, while the recited volume is desirable, some tolerances can occur when the volume is “substantially” the recited volume (e.g., 0.50 mL). Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, of +10% of the stated geometric construction, numerical value, and/or range.

As used herein, the terms “proximal” and “distal” refer to direction closer to and away from, respectively, an operator of the delivery device. Thus, for example, the end of the delivery device closest to or contacting the patient's body would be the distal end of the delivery device, while the end opposite the distal end (i.e., the end closer to the operator) would be the proximal end of the delivery device.

As used herein, the term “biomaterial component” (also referred to as “component”) includes any substance that is used in connection with any of the systems or delivery devices described herein to form a delivered biomaterial product. For example, a component can include a small molecule, catalyst, peptide, protein, enzyme, nucleotide (or derivatives of), short chains of nucleotides (or derivatives of), long chains of nucleotides (or derivatives of), monosaccharides (or derivatives of), disaccharides (or derivatives of), trisaccharides (or derivatives of), oligo saccharides (or derivatives of), polysaccharides (or derivatives of), monomer, oligomer, macromer, or polymer that can be cross-linked with another component to form a delivered product (e.g., hydrogel). A component can include a mixture or solution of one or more constituents (e.g., a polymer and a solvent). A component can include such constituents regardless of their state of matter (e.g., solid, liquid or gas). A component can include both active constituents and inert constituents. For example, in some embodiments, a component can include certain polymers that can form a delivered product, as well as a medicament or other active ingredient. By way of another example, in some embodiments a component can include drugs, including but not limited to, small molecule drugs and biologics. In other embodiments, a component can include certain constituents to impart desired properties to the delivered product, including constituents that facilitate the delivered product being echogenic, radiopaque, radiolucent, or the like.

The term “biomaterial product,” “delivered biomaterial product,” or “delivered product” includes any substance that is delivered by any of the systems or delivery devices described herein. For example, a delivered product can a biomaterial that is formed from multiple biomaterial components and delivered with any of the delivery systems described herein and then delivered to target locations. Thus, a delivered product can be the implant or structure that is formed with the system by multiple biomaterial components that react together or assemble into higher order structures via covalent and/or non-covalent bonds, and that is delivered by the system. For example, in certain situations, the delivered product can have a storage modulus (delivered G′) and a loss modulus (delivered G″) when the first component and the second component are conveyed out of a delivery member. The ratio of the delivered G″ to the delivered G′ can between about ⅓ and about 3. In some embodiments, the delivered G′ can be greater than the delivered G″ (i.e., the ratio of the delivered G″ to the delivered G′ is less than one), thus indicating that the delivered product is more solid than liquid. In some embodiments, the components can be formulated such that a viscoelastic substance (and not a liquid substance) is conveyed out of the exit opening of the delivery member.

In certain situations, the biomaterial can be delivered by the system in a substantially fully formed state to a target location. Although a delivered product can be considered substantially fully formed (i.e., the chemical reactions between the biomaterial components are substantially completed), it can still undergo certain changes (e.g., in vivo changes) after delivery. For example, a delivered biomaterial product can continue to absorb water and/or swell and/or can expel impurities. In some embodiments, a delivered biomaterial product can be a hydrogel that is formed by crosslinking of two or more biomaterial components. The hydrogel can be considered substantially fully formed when biomaterial no longer has small macromers (i.e., macromers of about 20,000 Daltons), exhibits observable viscoelastic properties as described above, and/or has no free end groups that have a size of about 20,000 Daltons or less. The term “hydrogel” can refer to any water-swollen (majority, >50%, of material mass is water), and cross-linked polymeric network produced by the reaction of one or more components (e.g., polymers, monomers) and/or a polymeric material that exhibits the ability to swell and retain a significant fraction of water within its structure but will not dissolve in water.

The term “gelation” refers to the transition of the hydrogel components from a soluble polymer of finite branches to a substance with infinitely large molecules. Similarly stated, “gelation” refers to the condition where the gel forms and after the components are combined. Thus, the gelation time refers to the time that it takes for the resulting hydrogel to substantially reach equilibrium.

As used herein, the term “set” can refer to multiple features or a singular feature with multiple parts. For example, when referring to set of walls, the set of walls can be considered as one wall with multiple portions, or the set of walls can be considered as multiple, distinct walls. Thus, a monolithically-constructed item can include a set of walls. Such a set of walls can include, for example, multiple portions that are either continuous or discontinuous from each other. A set of walls can also be fabricated from multiple items that are produced separately and are later joined together (e.g., via a weld, an adhesive, or any suitable method).

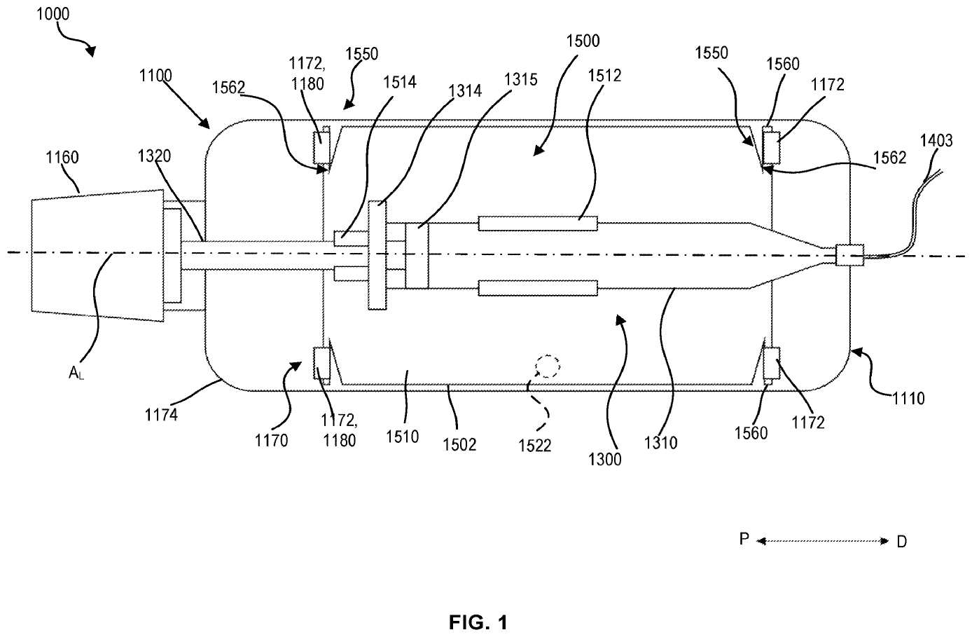

FIG. 1 is a schematic illustration of a portion of a delivery system 1000 according to an embodiment. As described herein, the delivery system 1000 is configured to convey a precise dose of composition, such as a biomaterial, to a target location. For example, the delivery system 1000 may be employed to deliver a formed hydrogel or other similar biomaterial within a body lumen. The delivery system 1000 includes a delivery device 1100 and a cartridge assembly 1500. The cartridge assembly 1500 includes a container 1300 supported by a cartridge chassis 1510.

The delivery device 1100 includes a housing 1110. The housing 1110 can be made from any suitable material or materials and contains a motive assembly (not shown in FIG. 1). The housing 1110 is configured to receive the cartridge chassis 1510 and secure the cartridge chassis 1510 in a fixed reference position relative to the housing 1110. In this manner, the cartridge chassis 1510 (or several different cartridge chassis of the same type) can be repeatedly coupled to the housing 1110 in the substantially the same position relative to the housing. By maintaining the cartridge chassis 1510 at the fixed reference position, the delivery device 1100 can repeatably deliver an accurate amount of the biomaterial from within the container 1300. Moreover, the housing 1110 is configured to secure the cartridge chassis 1510 in the fixed position, preventing any or all of movement in a longitudinal direction, a lateral direction, in a direction upwards from the housing, or a rotation direction during the delivery event. As described below, limiting movement of the cartridge chassis 1510 during delivery also improves the accuracy of the delivered biomaterial. In some embodiments, the housing 1110 can be constructed from multiple components that are joined together (e.g., via a hinged joint, a mechanical fastener or the like) to secure and/or at least partially surround the cartridge assembly 1500.

The motive assembly can be any suitable assembly or mechanism that produces a drive force to convey a composition from the container 1300. For example, the motive assembly may convey a first biomaterial component (also referred to as the first component), or a second biomaterial component (also referred to as the second component), or both the first component and the second component from the container 1300 as described herein. More specifically, the motive assembly can produce the drive force and/or convey the components within a desired velocity range, force range, and/or range of flow rates. By controlling the delivery characteristics of the composition, the delivery device 1100 can repeatably deliver an accurate amount of the component/delivered product (e.g., a formed hydrogel) to the target location. This, in turn, can lead to more consistent and improved outcomes. Additionally, controlling the delivery characteristics can also ensure that any desired reactions (e.g., a cross-linking reaction or other similar reactions resulting in a substantially solid mass) of the composition or between components of the composition are completed within the delivery system 1000. Said another way, controlling the delivery characteristics can ensure that the chemical reactions of the composition are substantially completed prior to contacting the target location. Controlling the delivery characteristics can also affect the size and/or shape of the delivered product and limits potential damage to the target tissue. Such damage can be caused by delivering an improper amount of the product or delivering the product too fast or at a force that causes tissue damage. Finally, controlling the delivery characteristics can also limit clogging or blockage within the delivery system 1000.

As shown, the delivery system 1000 includes a drive member 1160 that is coupled to the motive assembly and operably coupled to the container 1300. Therefore, when actuated, the motive assembly can move the drive member 1160 to convey the composition from the container 1300. The motive assembly can include any suitable mechanism for producing the drive force. For example, in some embodiments, the motive assembly can include an electromechanical driver (not shown in FIG. 1) to produce the drive force. Such electromechanical drivers can include, for example, a motor-driven linear actuator, a hydraulic actuator (e.g., that includes a pump driven by an electronic component), a magnetic-based actuator, a pneumatic actuator that includes an electromechanical valve to control a pressure applied to the drive member 1160, or any other suitable electromechanical driver of the types described herein. In some embodiments, the motive assembly and/or the delivery device 1100 can include an electronic control system (not shown) that controls the electromechanical driver and any other aspect of the motive assembly to control the delivery characteristics of the composition and/or the delivered product, as described herein. For example, in some embodiments, the electronic control system can be similar to the electronic control system 2200 described herein.

The motive assembly and the coupled drive member 1160 are configured to move the elastomeric member 1315 a predetermined travel distance at a predetermined rate in a distal direction (D). This movement results in the composition exiting the container 1300 at an exit velocity within a predetermined velocity range during over a specified time interval. The exit velocity and/or time interval may affect characteristics of the delivered/deposited product. For example, when depositing the product in a body lumen to form a plug, a fixed volume of the composition delivered at a first exit velocity may form a plug having a first length and a first density. However, when the fixed volume of the composition is delivered at a second, slower exit velocity, the resultant plug may have a second length that is shorter than the first length and a second density that is greater than the first density.

In some embodiments, the predetermined velocity range is bounded by an upper velocity threshold and a lower velocity threshold. During the deposition interval, the velocity of the components exiting the container 1300 (and/or exiting a delivery member 1403) is bounded by the upper velocity threshold and the lower velocity threshold. By maintaining the velocity below the upper velocity threshold, the composition can be delivered in a manner that that conforms to delivery site conditions (e.g., limits the likelihood of excessive velocity causing potential tissue damage). Moreover, maintaining the velocity below the upper velocity threshold can ensure that the delivered composition (e.g., biomaterial product) is properly formed within the delivery system 1000 (e.g., before exiting the delivery member). For example, if the delivered product has a gelation time of approximately 15 seconds, then the desired residence time of the composition within the delivery system 1000 is at least 15 seconds following actuation. Accordingly, the upper velocity threshold can be predetermined based on the length of the delivery member and the gelation time of the biomaterial product. The predetermined velocity range can be any suitable range to accommodate the desired delivery characteristics. For example, in some embodiments, the velocity range can be between 0.1 mm/sec to 10 mm/sec. In other embodiments, the velocity range can be between 0.1 mm/sec to 5 mm/sec. In other embodiments, the velocity range can be between 0.01 mm/sec and 1 mm/sec.

Moreover, by maintaining the velocity within the predetermined range and for the predetermined delivery time, the delivery system 1000 can deliver a volume of the composition that is within a desired volume range. In this manner, the amount of composition deposited at the target location can be accurately controlled. In some embodiments, the volume range is between about 5 microliters and about 1000 microliters. In other embodiments, the volume range is between about 50 microliters and 500 microliters. In yet other embodiments, the volume range is between about 50 microliters and 250 microliters. In still other embodiments, the volume range is between about 75 microliters and 150 microliters.

Additionally, in some embodiments, the lower velocity threshold is above zero. By maintaining the exit velocity (and/or the velocity within the delivery member) above zero, the likelihood of clogging within the delivery member is reduced.

In some embodiments a delivery device can include one or more feedback sensors to facilitate closed-loop control of the delivery characteristics of the composition. Such delivery characteristics can include the velocity of the components delivered from the container 1300, the velocity of the product delivered at the target site, the peak force applied during delivery, the amount of composition delivered, a sudden drop-off or spike in forces observed at the plunger, or the like.

The container 1300 includes an elastomeric member (or stopper) 1315 disposed within a container body 1310 to retain the composition within the container 1300. In other words, the container body 1310 defines a volume that is bounded on one side by the elastomeric member 1315 and that contains the composition. The container 1300 includes a plunger 1320 having an end portion movably disposed within the container body 1310 such that movement of the plunger 1320 in a distal direction (D) causes movement of the elastomeric member 1315 to convey the composition from the container 1300. The opposite end of the plunger 1320 is operably coupled to (e.g., is configured to engage) the drive member 1160. In some embodiments, the elastomeric member is made of a butyl rubber such as chlorobutyl or bromobutyl. In some embodiments, the elastomeric member can be coated with a film or other coatings such as ethylene tetrafluoroethylene (ETFE) or fluorinated ethylene propylene (FEP).

The container 1300 (and any of the containers described herein) can be any suitable container or set of containers. For example, the container 1300 can be a cartridge, an ampule, or a syringe. Moreover, the container body 1310 (and any of the container bodies described herein) can be of any suitable size and can be constructed from any suitable material such a type I borosilicate glass. As depicted, the container 1300 is coupled to a delivery member 1403 that can be any suitable delivery member, such as a needle, a catheter, or any other device through which the composition can be delivered to the target location. For example, the delivery member 1403 may be inserted into a body lumen to deposit the composition (e.g., the biomaterial product) therein. The body lumen can be any suitable body lumen, such as, for example, an artery, vein, capillary, vessel, tissue, intra-organ space, lymphatic vessel, vas deferens, epididymis, fallopian tube, duct, bile duct, hepatic duct, cystic duct, pancreatic duct, parotid duct, organ, uterus, prostate. The target location can also be an organ of a gastrointestinal tract or circulatory system or respiratory system or nervous system, a subcutaneous space, an intramuscular space, or an interstitial space.

In some embodiments, the composition can include the first component and a second component. The first and second components can be any of the biomaterial components described herein. For example, in some embodiments, the first component and the second component can each be a water-soluble component (e.g., monomer, macromer, polymer, or the like) that is capable of crosslinking (e.g., with the other component) to form a hydrogel (as the delivered biomaterial product). In some embodiments, the first component and the second component are formulated such that the resulting hydrogel has a gelation time of less than 5 minutes. In other embodiments, the first component and the second component are formulated such that the resulting hydrogel has a gelation time of less than 2 minutes. In other embodiments, the first component and the second component are formulated such that the resulting hydrogel has a gelation time of less than 1 minute. In yet other embodiments, the first component and the second component are formulated such that the resulting hydrogel has a gelation time of less than 30 seconds. In some embodiments, the first component is at least one of a polyvinyl alcohol, alginate or modified alginate, chitosan or modified chitosan, polyethyleneimine, carboxymethyl cellulose, and/or polyethylene glycol terminated with a biorthogonal functional group (e.g., amine, thiol, maleimide, azide, activated ester). The second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group (e.g., amine, thiol, maleimide, azide, activated ester). In some embodiments, polyvinyl alcohol, alginate, chitosan, polyethyleneimine, carboxymethyl cellulose, polyethylene glycol terminated with functional groups, divalent cations, reduced hyaluronic acid, polystyrene sulfonate, or gelatin have a weight percent ranging from about 1 to 30% in solvent. In some embodiments the polysaccharides may be modified with different functional groups. In some embodiments the polysaccharides and proteins may range in molecular weight from 10,000-1,000,000 grams/mole. In some embodiments, the polyvinyl alcohol, polystyrene sulfonate, polyethyleneimine, and polyethylene glycol may be linear, Y-shaped, 3-arm, 4-arm, 6-arm, or 8-arm and range in molecular weight from 1,000-1,000,000 grams/mole. The hydrogel can be any of the hydrogels described herein and can have any of the characteristics as indicated herein. For example, in some embodiments, the formed hydrogel can be at least 90 percent water.

As depicted, the housing 1110 defines a coupling interface 1170 that is configured to receive the cartridge chassis 1510. The coupling interface 1170 includes at least one longitudinal positioning feature 1172 (four longitudinal positioning features are shown in FIG. 1). The longitudinal positioning feature(s) 1172 is configured to engage a corresponding longitudinal positioning feature 1560 of the cartridge chassis 1510 and establish a longitudinal position of the cartridge chassis 1510. In some embodiments, at least one longitudinal positioning feature 1172 can be configured as a proximal retaining portion 1180. Similarly stated, the longitudinal positioning feature(s) 1172 and the corresponding longitudinal positioning feature(s) 1560 of the cartridge chassis 1510 are configured to secure the chassis 1510 at a reference position relative to the housing 1110 and to limit (or prevent) movement of the chassis 1510 relative to the housing 1110. Specifically, the longitudinal positioning feature(s) 1172 and the corresponding longitudinal positioning feature(s) 1560 can limit (or prevent) movement of the chassis 1510 relative to the housing 1110 along a longitudinal axis AL. The housing 1110 also defines a mounting surface 1174 of the coupling interface 1170 that has a maximal dimension that extends between the distal direction (D) and the proximal direction (P).

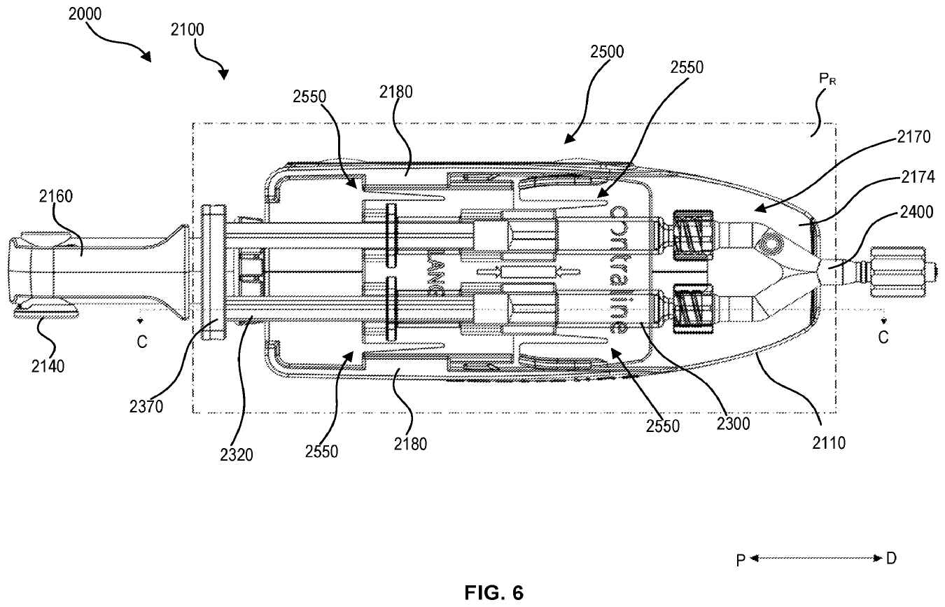

In some embodiments, the cartridge chassis 1510 includes one ore more coupling members 1550. For example, as depicted in FIG. 1, the cartridge chassis 1510 can include four coupling members 1550 to establish one or more points of securement with the housing 1110 (four points of attachment are shown in FIG. 1). However, in some embodiments the cartridge chassis 1510 can include two coupling members 1550, three coupling members 1550, or more than four coupling members 1550. The coupling members 1550 are distributed about a perimeter 1502 of the cartridge chassis 1510 and are positioned to be received by the coupling interface 1170 of a delivery device 1100, as described above. As described below, one or more of the coupling members 1550 can include a longitudinal positioning feature 1560, a living hinge 1562, or other suitable structure to facilitate easy coupling and decoupling of the cartridge chassis 1510 to the housing. As depicted, in some embodiments, the coupling interface 1170 receives the plurality of coupling members 1550 in a proximal-distal configuration. However, in additional embodiments, the coupling interface 1170 receives the plurality of coupling members 1550 in a side-side configuration (such as depicted in FIG. 6).

At least one coupling member 1550 includes a longitudinal positioning feature 1560 positioned to engage a corresponding feature (e.g., longitudinal positioning feature 1172) of the coupling interface 1170 of the delivery device 1100. This engagement prevents longitudinal movement of the cartridge chassis 1510 relative to the coupling interface 1170. Additionally, the cartridge chassis 1510 includes a container clip 1512 that, at least partially, surrounds the container body 1310 and secures the container 1300 in a fixed position relative to the cartridge chassis 1510. Accordingly, when the cartridge assembly 1500 is coupled to the delivery device 1100, the container 1300 is established in a fixed position relative to the delivery device 1100.

At least one coupling member 1550 includes a living hinge 1562 that is oriented to exert a force on the delivery device 1100 (e.g., on the housing 1110) when the cartridge assembly 1500 is received by the coupling interface 1170. For example, as depicted in FIG. 1, each living hinge 1562 of the coupling members 1550 is oriented to exert a longitudinal force in either the proximal (P) or distal (D) direction on the housing 1110. Accordingly, the coupling member(s) 1550 and/or the longitudinal positioning feature(s) 1560 have an extended position or a neutral position when the cartridge chassis 1510 is separated from the delivery device 1100 and a compressed position when received by the coupling interface 1170. In some embodiments, the engagement between at least one coupling member 1550 and the coupling interface 1170 also develops a force that is substantially orthogonal to the mounting surface 1174. In this manner, the cartridge chassis 1510 can be secured to the mounting surface 1174 and movement away from the mounting surface (i.e., in a direction substantially orthogonal to the mounting surface 1174) can be limited or prevented. As described above, by limiting (or preventing) movement of the cartridge chassis 1510 relative to the housing 1110 during use, the accuracy of the delivery of materials from within the container 1300 can be improved.

As depicted, in some embodiments, the cartridge chassis 1510 includes an optional plunger guide structure 1514. The plunger guide structure 1514 is in axial alignment with the container clip 1512. The plunger guide structure 1514 is positioned to at least partially circumscribe the plunger 1320 of the container 1300. When the container 1300 is secured by the container clip 1512, the flange 1314 abuts the plunger guide structure 1514. Accordingly, the plunger guide structure 1514 limits a longitudinal movement of the container 1300 in the proximal (P) direction. In some embodiments, the cartridge chassis 1510 need not include a plunger guide structure 1514 as shown, but can instead include a flange interface (not shown) that does not surround the plunger 1320. In such embodiments, the flange interface (not shown) can abut the flange 1314 or any other portion of the container 1300 to limit (or prevent) movement of the container 1300 along the longitudinal axis AL. In yet other embodiments, the cartridge chassis 1510 does not include either the plunger guide structure 1514 or a flange interface.

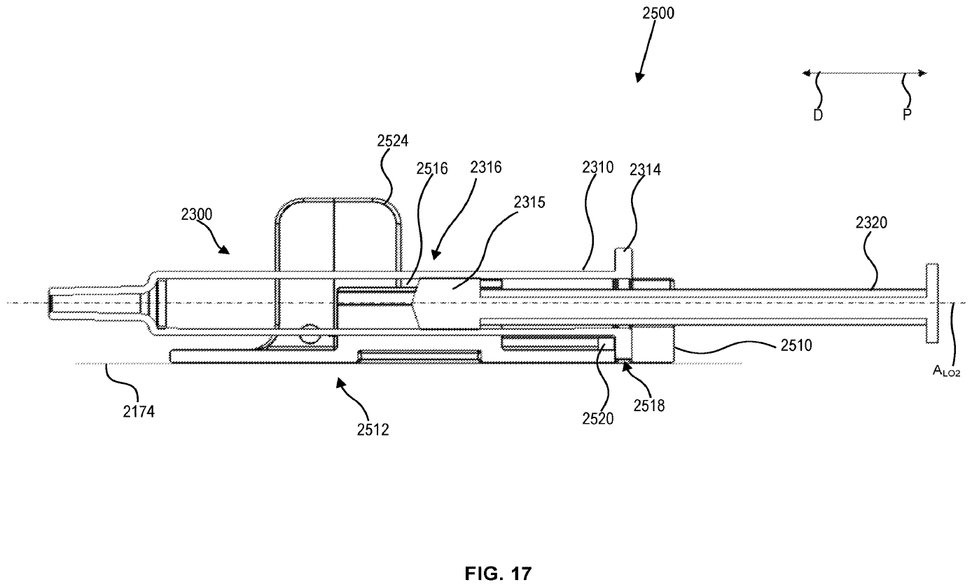

In some embodiments, the container clip 1512 (e.g., any container clip described herein) exerts a retention force on the container body 1310. The retention force is directed toward the longitudinal axis (e.g., longitudinal axis (A_{LO2}) as depicted in FIG. 17) of the container 1300. The retention force may have a maximal magnitude that corresponds to a release threshold at which a retained container body 1310 is released from the container clip. In other words, when a force acting on the container 1300 exceeds the release threshold, the container is released from the cartridge chassis 1510. Accordingly, the release threshold establishes a maximal force that can be applied to the composition via the elastomeric member 1315 and, thus, corresponds to a maximal force that can be applied at the target location by the composition. For example, in some embodiments, the container clip 1512 can be configured to release the container body 1310 when a force greater than five pounds is exerted on the container 1300. In additional embodiments, the container clip 1512 can be configured to release the container body 1310 when a force between five pounds and seven pounds is exerted on the container 1300. In further embodiments, the container clip 1512 can be configured to release the container body 1310 when a force between seven pounds and ten pounds is exerted on the container 1300. In yet further embodiments, the container clip 1512 can be configured to release the container body 1310 when a force greater than ten pounds is exerted on the container 1300.

In some embodiments, the cartridge chassis 1510 includes an optional sensor interface feature 1522. The sensor interface feature 1522 is configured to operably engage with the positional sensor (not shown) of the delivery device 1100. The positional sensor can be a photoelectric sensor, an ultrasonic position sensor, an inductive proximity sensor, an electrical contact sensor, a magnetic contact sensor, a reflective laser sensor, a compression sensor, or other similar sensor. In some embodiments, the sensor interface feature 1522 can be sized to preclude the engagement of the positional sensor on the condition that the cartridge chassis 1510 deviates from a nominal longitudinal and/or lateral position. For example, the sensor interface feature 1522 can be sized to engage the position sensor by occluding a photoelectric sensor only when the cartridge chassis 1510 is in the nominal longitudinal position. This arrangement allows the sensor to produce a signal indicating either proper alignment or misalignment of the cartridge chassis 1510. The sensor interface feature 1522 can any suitable feature that can operably engage with the sensor, such as, for example, an opening, a transparent region of the cartridge chassis 1510, a magnetic portion, a reflective region of the cartridge chassis 1510, or a colored region of the cartridge chassis.

In some embodiments, the cartridge assembly can include structures and features to ensure that a container coupled thereto is in the nominal (or reference) position relative to the delivery device throughout the operation of the delivery system. By using a cartridge that includes indexing and/or alignment features, the likelihood of misalignment and/or improper positioning by the user can be minimized. Moreover, such an arrangement allows for multiple different cartridges to used on the same delivery device while preserving accuracy of delivery.

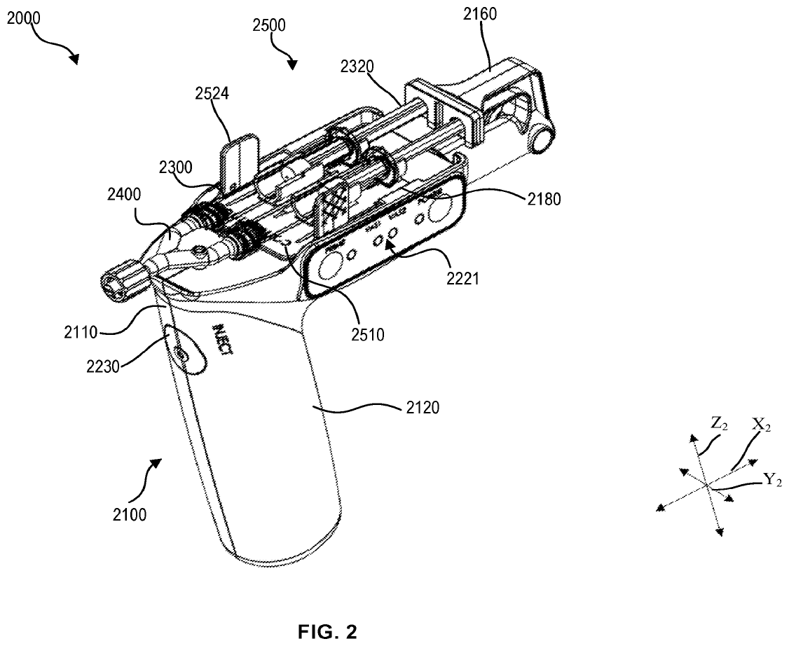

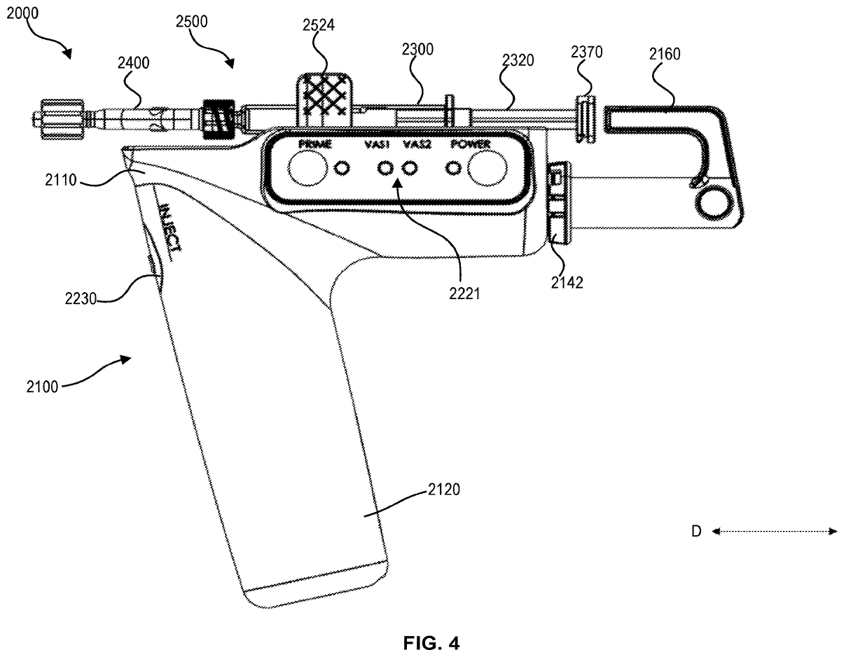

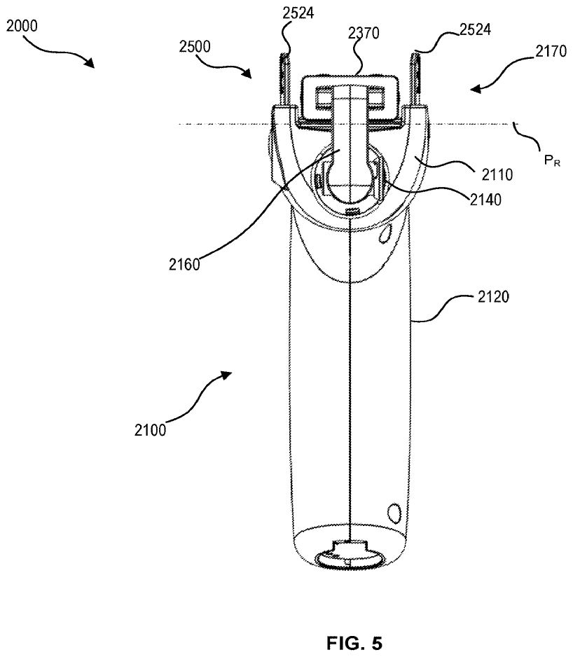

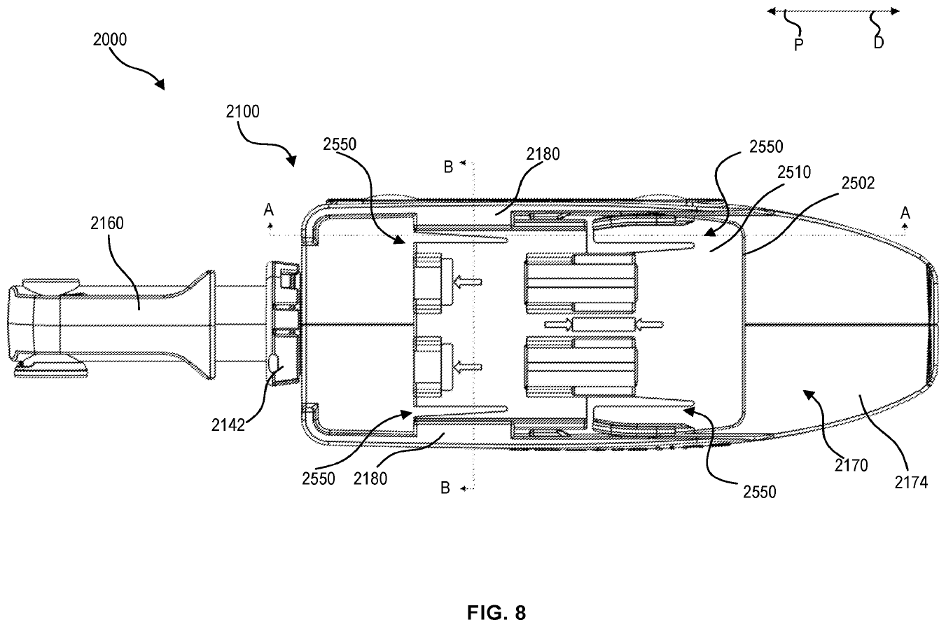

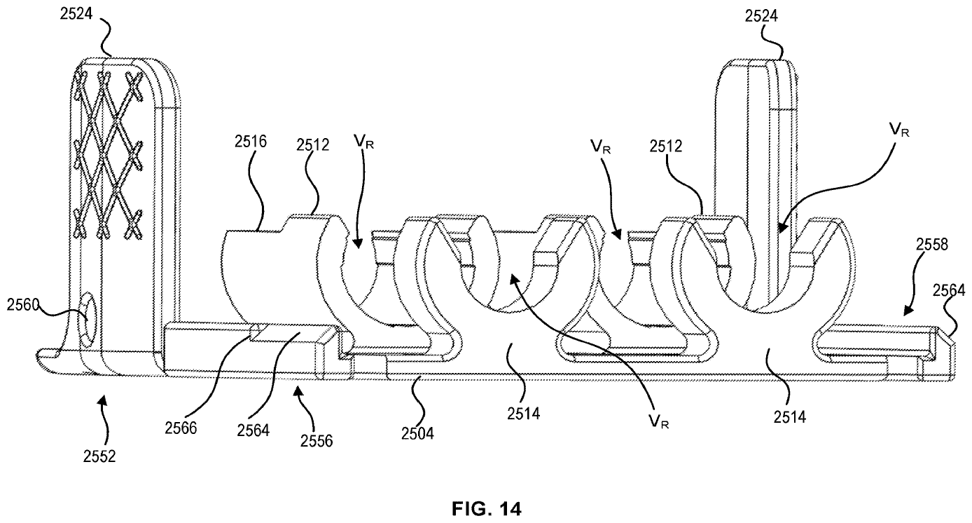

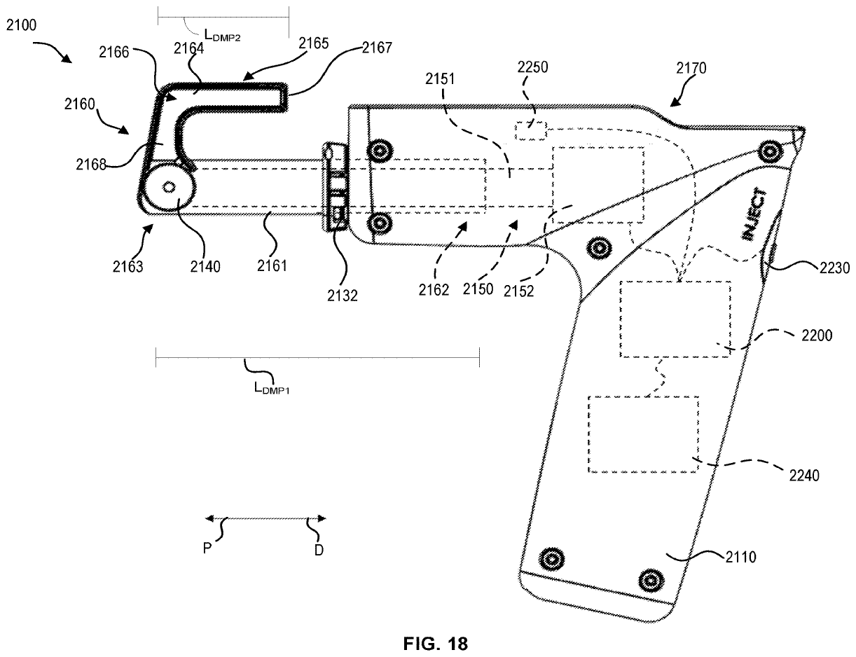

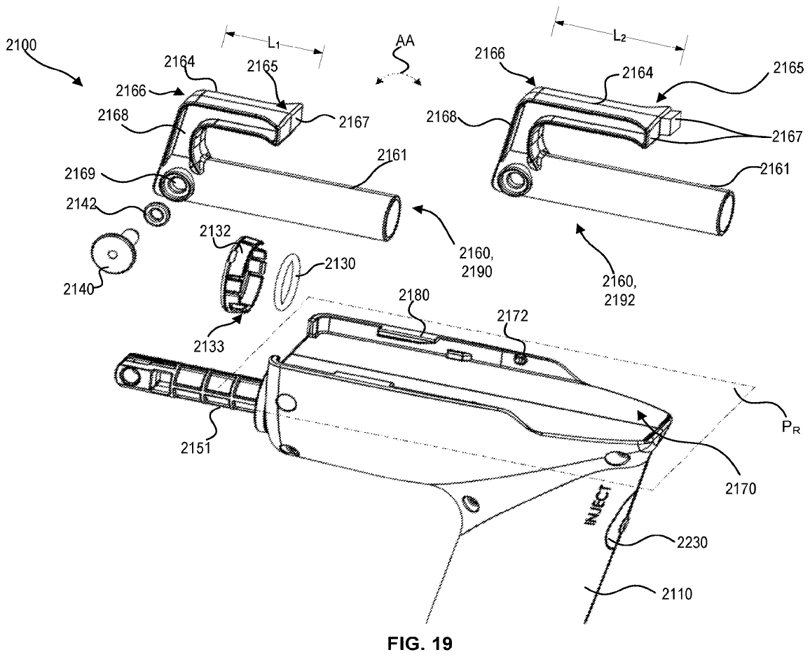

FIGS. 2-19 are various views of a delivery system 2000 according to an embodiment, that includes, among other things, a cartridge chassis for ensuring that the container is repeatably maintained in a fixed position relative to a delivery device. As described herein, the delivery system 2000 is configured to convey a precise and/or accurate dose of composition, such as a biomaterial, to a target location. The methods described herein and any of the hydrogel formulations or systems described herein can be similar to those described in U.S. Pat. No. 11,318,040, entitled “Systems and Methods for Delivering of Biomaterials,” filed on Nov. 12, 2019, and International Patent Publication No. WO2021/231717, entitled “Biomaterial Compositions and Methods of Delivery,” filed on May 13, 2021, each of which is incorporated herein by reference in its entirety. For example, the delivery system 2000 may be employed to deliver a formed hydrogel or other similar biomaterial within a body lumen. Accordingly, the system 2000 includes the delivery device 2100 (e.g., FIGS. 2-6, 8-12, 18, and 19) and a cartridge assembly 2500 (e.g., FIGS. 2-6). The delivery device 2100 includes a housing 2110, a motive assembly 2150 (e.g., FIGS. 18 and 19) and an electronic control system 2200 (e.g. FIG. 18). The cartridge assembly 2500 includes a container 2300 (which includes two container bodies) supported by a cartridge chassis 2510.

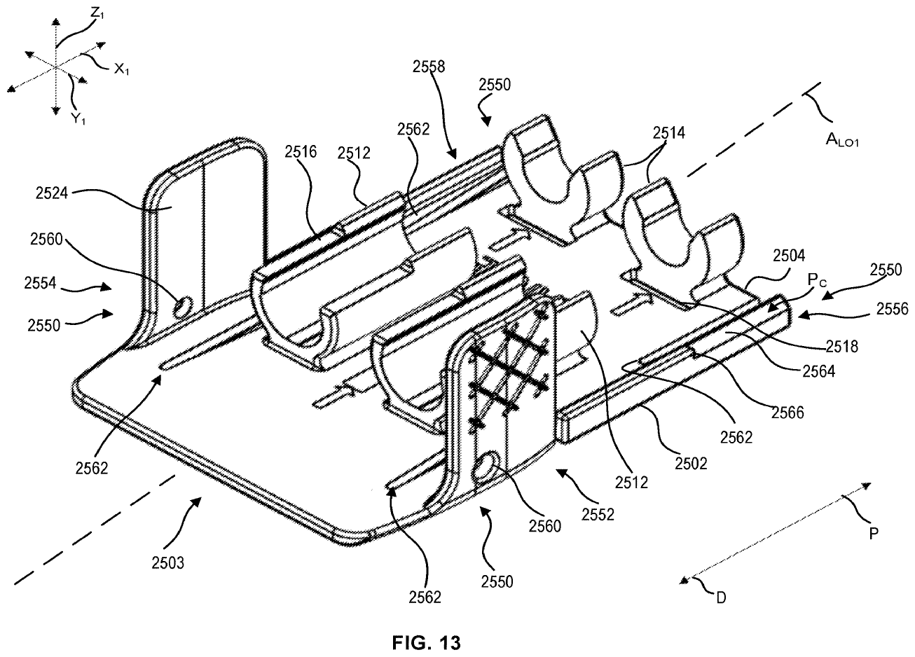

The delivery device 2100 includes a housing 2110. The housing 2110 can be made from any suitable material or materials. As shown, the housing 2110 includes a handle 2120 that can be gripped and/or manipulated by a user during operation of the delivery device 2100. A motive assembly 2150 (e.g., FIG. 18) is contained within the housing 2110. Additionally, the housing 2110 is configured to receive the cartridge chassis 2510 and secure the cartridge chassis 2510 in a fixed position (e.g., in a fixed position along the longitudinal axis x₁, see FIG. 13) relative to the housing 2110. In some embodiments, the housing 2110 can be constructed from multiple components that are joined together (e.g., via a hinged joint, a mechanical fastener or the like) to secure and/or at least partially surround the cartridge assembly 2500.

The motive assembly 2150 can be any suitable assembly or mechanism that produces a drive force to convey a composition from the container 2300. For example, the motive assembly 2150 may convey a first biomaterial component (also referred to as the first component), or a second biomaterial component (also referred to as the second component), or both the first component and the second component from the container 2300 as described herein. More specifically, the motive assembly 2150 can produce the drive force and/or convey the components within a desired velocity range, force range, and/or range of flow rates. By controlling the delivery characteristics of the composition, the delivery device 2100 can repeatably deliver an accurate amount of the component/delivered product (e.g., a formed hydrogel) to the target location. This, in turn, can lead to more consistent and improved outcomes. Additionally, controlling the delivery characteristics can also ensure that any desired reactions (e.g., a cross-linking reaction or other similar reactions resulting in a substantially solid mass) of the composition or between components of the composition are completed within the delivery system 2000. Said another way, controlling the delivery characteristics can ensure that the chemical reactions of the composition are substantially completed prior to contacting the target location. Controlling the delivery characteristics can also affect the size and/or shape of the delivered product and limits potential damage to the target tissue. Such damage can be caused by delivering an improper amount of the product or delivering the product too fast or at a force that causes tissue damage. Finally, controlling the delivery characteristics can also limit clogging or blockage within the delivery system 2000.

As shown, the delivery system 2000 includes a drive member 2160 that is coupled to an output rod 2151 of the motive assembly 2150 and operably coupled to the container 2300. Therefore, actuation, the motive assembly 2150 can move the drive member 2160 to convey the composition from the container 2300. The motive assembly 2150 can include any suitable mechanism for producing the drive force. For example, in some embodiments, the motive assembly can include an electromechanical driver 2152 (e.g., FIG. 18) to produce the drive force. Such electromechanical drivers 2152 can include, for example, a motor-driven linear actuator, a hydraulic actuator (e.g., that includes a pump driven by an electronic component), a magnetic-based actuator, a pneumatic actuator that includes an electromechanical valve to control a pressure applied to the drive member 2160, or any other suitable electromechanical driver of the types described herein.

The motive assembly 2150 and the coupled drive member 2160 are configured to move the elastomeric member 2315 a predetermined travel distance at a predetermined rate in a distal direction (D). This movement results in the composition exiting the container 2300 at an exit velocity within a predetermined velocity range during over a specified time interval. The exit velocity and/or time interval may affect characteristics of the delivered/deposited product. For example, when depositing the product in a body lumen to form a plug, a fixed volume of the composition delivered at a first exit velocity may form a plug having a first length and a first density. However, when the fixed volume of the composition is delivered at a second, slower exit velocity, the resultant plug may have a second length that is shorter than the first length and a second density that is greater than the first density.

In some embodiments, the predetermined velocity range is bounded by an upper velocity threshold and a lower velocity threshold. During the deposition interval, the velocity of the components exiting the container 2300 (and/or exiting a delivery member (not shown)) is bounded by the upper velocity threshold and the lower velocity threshold. By maintaining the velocity below the upper velocity threshold, the composition can be delivered in a manner that that conforms to delivery site conditions (e.g., limits the likelihood of excessive velocity causing potential tissue damage). Moreover, maintaining the velocity below the upper velocity threshold can ensure that the delivered composition (e.g., biomaterial product) is properly formed within the delivery system 2000 (e.g., before exiting the delivery member). For example, if the delivered product has a gelation time of approximately 15 seconds, then the desired residence time of the composition within the delivery system 2000 is at least 15 seconds following actuation. Accordingly, the upper velocity threshold can be predetermined based on the length of the delivery member and the gelation time of the biomaterial product. The predetermined velocity range can be any suitable range to accommodate the desired delivery characteristics. For example, in some embodiments, the velocity range can be between 0.1 mm/sec to 10 mm/sec. In other embodiments, the velocity range can be between 0.1 mm/sec to 5 mm/sec. In other embodiments, the velocity range can be between 0.01 mm/sec and 1 mm/sec.

Moreover, by maintaining the velocity within the predetermined range and for the predetermined delivery time, the delivery system 2000 can deliver a volume of the composition that is within a desired volume range. In this manner, the amount of composition deposited at the target location can be accurately controlled. In some embodiments, the volume range is between about 5 microliters and about 1000 microliters. In other embodiments, the volume range is between about 50 microliters and 500 microliters. In yet other embodiments, the volume range is between about 50 microliters and 250 microliters. In still other embodiments, the volume range is between about 75 microliters and 150 microliters.

Additionally, in some embodiments, the lower velocity threshold is above zero. By maintaining the exit velocity (and/or the velocity within the delivery member) above zero, the likelihood of clogging within the delivery member is reduced.

In some embodiments a delivery device can include one or more feedback sensors to facilitate closed-loop control of the delivery characteristics of the composition. Such delivery characteristics can include the velocity of the components delivered from the container 2300, the velocity of the product delivered at the target site, the peak force applied during delivery, the amount of composition delivered, a sudden drop-off or spike in forces observed at the plunger, or the like.

As depicted in FIGS. 18 and 19, in some embodiments, the drive member 2160 is removably coupled to the motive assembly 2150. In this manner, the drive member 2160 can be easily cleaned and/or sterilized separately from the remainder of the delivery device 2100 after each use. Thus, the removable drive member 2160 can facilitate establishing and maintaining a sterile field associated with the system 2000 in a manner that limits the likelihood of contamination or other compromise of the sterile field. Specifically, as described herein, the drive member 2160 is coupled to the output rod 2151 and the remainder of the delivery device 2100 via various sealed couplings to limit the likelihood that pathogens or microbes can pass between regions within the housing 2110 and regions outside of the housing during a procedure. Thus, the outer surfaces of the housing 2110 can be wiped, cleaned, or otherwise sterilized in a manner that will not damage the internal components (e.g., electronics). The drive member 2160, however, can be subjected to a separate cleaning or sterilization protocol. For example, the drive member 2160 can be constructed from materials and otherwise configured to be used with any suitable sterilization technique or medium, including ethylene oxide, gamma radiation, e-beam radiation, ultraviolet radiation, steam, plasma, or hydrogen peroxide.

Additionally, by having a removable drive member 2160, the system 2000 can include different drive members having different dimensions to facilitate different delivery profiles. For example, as described in more detail below, the system 2000 can include drive members 2160 having different lengths that result in different amounts of materials being delivered for a given movement of the motive assembly 2150.

As shown, in FIGS. 18 and 19, the drive member 2160 includes a first drive member portion 2161 that is coupled to the output rod 2151 and a second drive member portion 2164. A first distal end 2162 of the first drive member portion 2161 is positioned within the housing 2110. During use, the output rod 2151 moves when the motive assembly 2150 is actuated causing the first distal end 2162 to be moved into (or out of) the housing 2110. A second distal end 2165 of the second drive member portion 2164 is configured to be operatively coupled to the container (e.g., to engage the plunger or the plunger link 2370) to move the elastomeric member within the container body. Accordingly, the second distal end 2165 is formed as a contact face 2167 that is configured to engage the plunger of the container.

As depicted, the first drive member portion 2161 has a longitudinal length (L_{DMP1}) that extends between the first distal end 2162 and a first proximal end 2163. The second drive member portion 2164 is oriented parallel to the first drive member portion 2161. The second drive member portion 2164 has a longitudinal length (L_{DMP2}) extending between the second distal end 2165 and a second proximal end 2166. The longitudinal length (L_{DMP2}) of the second drive member portion 2164 establishes a home (e.g., initial) position of the elastomeric member and/or a travel distance of the elastomeric member relative to the housing 2110 within the container for a given stroke length of the motive assembly 2150.

The drive member 2160 also includes a proximal joining member (or portion) 2168 that is coupled between the first proximal end 2163 and the second proximal end 2166. In other words, the first drive member portion 2161 and the second drive member portion 2164 are coupled via the proximal joining member 2168. In some embodiments, the proximal joining member 2168 has a length (e.g. a length along the axis z₂ of FIG. 2) that establishes the second drive member portion 2164 in a parallel orientation relative to the first drive member portion 2161. Additionally, the length of the proximal joining member 2168 can position the second drive member portion in axial alignment with the container 2300.

As depicted in FIG. 19, in some embodiments, the drive member 2160 can, as indicated by arrow AA, be interchanged with an additional drive member having a different structure and/or dimensions. Accordingly, the delivery device 2100 can include a first interchangeable drive member 2190 and at least a second interchangeable drive member 2192. The drive members are described herein with reference to the drive member 2160 and its structure. The first interchangeable drive member 2190 and the second interchangeable 2192 shown in FIG. 19 can be similar to (or the same as) the general drive member 2160 described herein, but are identified separately because they may have slight differences (e.g., different lengths associated with different delivery characteristics or different structure of the contact face 2167). The longitudinal length (e.g., longitudinal length (L_{DMP2}) as depicted in FIG. 18) of the second drive member portion 2164 of the first interchangeable drive member 2190 has a first longitudinal length (L₁). The first longitudinal length (L₁) establishes a first home (e.g., initial) position of the elastomeric member and/or a first travel distance of the elastomeric member relative to the housing 2110 for a given stroke length of the motive assembly 2150. The longitudinal length (e.g., longitudinal length (L_{DMP2}) as depicted in FIG. 18) of the second drive member portion 2164 of the second interchangeable drive member 2192 has a second longitudinal length (L₂). The second longitudinal length (L₂) is different than the first longitudinal length (L₁). Accordingly, the second longitudinal length (L₂) establishes a second home (e.g., initial) position of the elastomeric member and/or a second travel distance of the elastomeric member relative to the housing 2110 for the same stroke length of the motive assembly 2150. The second travel distance of the elastomeric member is different than the first travel distance of the elastomeric member.

As depicted in FIGS. 18 and 19, in some embodiments, a first sealing member 2130 circumscribes the first drive member portion 2161. The first sealing member 2130 is positioned between the first distal end 2162 and the first proximal end 2163 of the first drive member portion 2161. The first sealing member 2130 can, for example, be an O-ring or other similar structure formed from a suitable elastomer. During operation of the delivery system 2000, the first sealing member 2130 remains in a fixed position relative to the housing 2110 while the first drive member portion 2161 slides within the first sealing member 2130.

A sealing clamp ring 2132 circumscribes the first sealing member 2130 and the first drive member portion 2161. The sealing clamp ring 2132 is positioned between the first sealing member 2130 and the first proximal end 2163 of the first drive member portion 2161. The sealing clamp ring 2132 is configured to exert a compressive force on the first sealing member 2130. The compressive force against the first sealing member 2130 establishes a fluidic seal between the first drive member portion 2161 and the housing 2110. In other words, the first sealing member 2130 forms a substantially fluid-tight seal between the drive member 2160 and the housing 2110. The substantially fluid-tight seal precludes entry of contaminants or other fluids into the housing 2110, thus facilitating the reusability of the delivery device 2100. In some embodiments, the removal of the sealing clamp ring 2132 may be desirable in response to certain conditions (e.g., to facilitate easy removal of drive member 2160 (e.g., for cleaning). Accordingly, the sealing clamp ring 2132 defines a set of deformable protrusions having locking tabs, a set of grooves between the protrusions, and a set of decoupling recesses 2133. When the sealing clamp ring 2132 is coupled to the housing, the deformable protrusions are deformed outward around a shoulder of the housing, and then the locking tabs secure the sealing clamp ring 2132 to the shoulder. The set of decoupling recesses 2133 are distributed about the circumference of the sealing clamp ring 2132 and are aligned with each of the deformable protrusions. The plurality of decoupling recesses 2133 are sized to receive a portion of a removal instrument (not shown). The removal instrument can be placed through the decoupling recesses 2133 and can urge the deformable protrusions further outward (away from the shoulder) to allow removal of the sealing clamp ring 2132.

As further depicted in FIGS. 18 and 19, in some embodiments, the drive member 2160 is removably coupled to the motive assembly 2150 by a removable retaining pin 2140. The removable retaining pin 2140 is positioned within a retention orifice 2169 of the first drive member portion 2161 and a corresponding opening within the output rod 2151 of the motive assembly 2150. A second sealing member 2142 circumscribes the removable retaining pin 2140. The second sealing member 2142 is positioned within a groove surrounding the retention orifice 2169 to fluidically seal (i.e., produce a substantially fluid-tight seal) the retention orifice 2169. The substantially fluid-tight seal precludes entry of contaminants or other fluids into the interior of the drive member 2160 and the housing 2110 via the first drive member portion 2161, thus facilitating the reusability of the delivery device 2100 while retaining the ability to interchange the first interchangeable drive member 2190 and at least the second interchangeable drive member 2192 and/or performing maintenance operation on the delivery device 2100. The second sealing member 2142 can, for example, be an O-ring or other similar structure formed from a suitable elastomer.

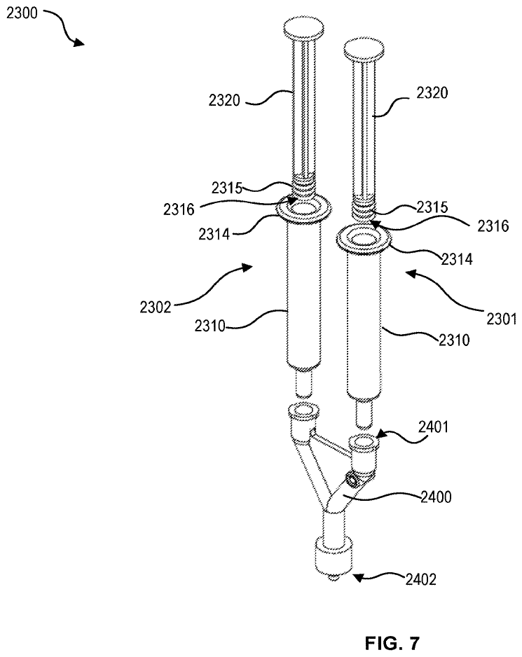

Referring to FIG. 7, each container 2300 includes an elastomeric member (or stopper) 2315 disposed within a container body 2310 to retain the composition within the container 2300. In other words, the container body 2310 defines a volume that is bounded on one side by the elastomeric member 2315 and that contains the composition. The container 2300 includes a plunger 2320 having an end portion movably disposed within the container body 2310 such that movement of the plunger 2320 in a distal direction (D) causes movement of the elastomeric member 2315 to convey the composition from the container 2300. The opposite end of the plunger 2320 is operably coupled to (e.g., is configured to engage) the drive member 2160 via a plunger link 2370. In some embodiments, the elastomeric member 2315 is made of a butyl rubber such as chlorobutyl or bromobutyl. In some embodiments, the elastomeric member 2315 can be coated with a film or other coatings such as ethylene tetrafluoroethylene (ETFE) or fluorinated ethylene propylene (FEP).

The container 2300 (and any of the containers described herein) can be any suitable container. For example, the container 2300 can be a cartridge, an ampule, or a syringe. Moreover, the container body 2310 (and any of the container bodies described herein) can be of any suitable size and can be constructed from any suitable material such a type I borosilicate glass. The container 2300 is coupled to a delivery member (not shown) that can be any suitable delivery member, such as a needle, a catheter, or any other device through which the composition can be delivered to the target location. For example, the delivery member may be inserted into a body lumen to deposit the composition (e.g., the biomaterial product) therein. The body lumen can be any suitable body lumen, such as, for example, an artery, vein, capillary, vessel, tissue, intra-organ space, lymphatic vessel, vas deferens, epididymis, fallopian tube, duct, bile duct, hepatic duct, cystic duct, pancreatic duct, parotid duct, organ, uterus, prostate. The target location can also be an organ of a gastrointestinal tract or circulatory system or respiratory system or nervous system, a subcutaneous space, an intramuscular space, or an interstitial space. In some embodiments, the delivery member can be and any of the catheters described in U.S. Pat. No. 11,318,040, entitled “Systems and Methods for Delivering of Biomaterials,” filed on Nov. 12, 2019, and International Patent Publication No. WO2021/231717, entitled “Biomaterial Compositions and Methods of Delivery,” filed on May 13, 2021, each of which is incorporated herein by reference in its entirety.

In some embodiments, the composition can include the first component and a second component. The first and second components can be any of the biomaterial components described herein. For example, in some embodiments, the first component and the second component can each be a water-soluble component (e.g., monomer, macromer, polymer, or the like) that is capable of crosslinking (e.g., with the other component) to form a hydrogel (as the delivered biomaterial product). In some embodiments, the first component and the second component are formulated such that the resulting hydrogel has a gelation time of less than 5 minutes. In other embodiments, the first component and the second component are formulated such that the resulting hydrogel has a gelation time of less than 2 minutes. In other embodiments, the first component and the second component are formulated such that the resulting hydrogel has a gelation time of less than 1 minute. In yet other embodiments, the first component and the second component are formulated such that the resulting hydrogel has a gelation time of less than 30 seconds. In some embodiments, the first component is at least one of a polyvinyl alcohol, alginate or modified alginate, chitosan or modified chitosan, polyethyleneimine, carboxymethyl cellulose, and/or polyethylene glycol terminated with a biorthogonal functional group (e.g., amine, thiol, maleimide, azide, activated ester). The second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group (e.g., amine, thiol, maleimide, azide, activated ester). In some embodiments, polyvinyl alcohol, alginate, chitosan, polyethyleneimine, carboxymethyl cellulose, polyethylene glycol terminated with functional groups, divalent cations, reduced hyaluronic acid, polystyrene sulfonate, or gelatin have a weight percent ranging from about 1 to 30% in solvent. In some embodiments the polysaccharides may be modified with different functional groups. In some embodiments the polysaccharides and proteins may range in molecular weight from 10,000-1,000,000 grams/mole. In some embodiments, the polyvinyl alcohol, polystyrene sulfonate, polyethyleneimine, and polyethylene glycol may be linear, Y-shaped, 3-arm, 4-arm, 6-arm, or 8-arm and range in molecular weight from 1,000-1,000,000 grams/mole. The hydrogel can be any of the hydrogels described herein and can have any of the characteristics as indicated herein. For example, in some embodiments, the formed hydrogel can be at least 90 percent water.

As depicted in FIGS. 2, 3, 6, and 7, in some embodiments, the cartridge assembly 2500 can include more than one container 2300. For example, the cartridge assembly 2500 can include a first container 2301 containing the first component of the composition. The cartridge assembly 2500 can also include a second container 2302. The first container 2301 and the second container 2302 can be retained by the cartridge chassis 2510 so that the longitudinal axis (A_{LO2}) (e.g., FIG. 17) of the second container 2302 is parallel to the longitudinal axis (A_{LO2}) of the first container 2301. Said another way, the first and second containers 2301, 2302 are arranged in a side-by-side configuration and coplanar. The first container 2301 and the second container 2302 are configured to be coupled to the connector 2400, which is, in turn, configured to be coupled to a deliver member (not shown; e.g., a catheter or needle). By having the containers as separate articles from the connector, the first container 2301 and the second container 2302 can be commercially available containers (e.g., syringes) within which the first component and the second component, respectively, can be prepared for use. Moreover, this arrangement allows the first component to be prepared within the first container 2301 (e.g., via mixing, dilution, etc.) separately from when the second component is prepared within the second container 2302. In other embodiments, however, the container 2300 can include a first container and a second container that are integrally and/or monolithically constructed with the connector. In yet other embodiments, the container 2300 can include a single container that contains both the first component and the second component.

In some embodiments, the plungers 2320 of the first container 2301 and the second container 2302 are coupled via a plunger link 2370 of the cartridge assembly 2500. Specifically, the plunger link 2370 defines a retention slot and includes a surface against which the contact face 2167 of the drive member 2160 can exert a drive force. The retention slot receives a portion of the plungers 2320. The plunger link 2370 facilitates the use of a single motive assembly 2150 and single drive member 2160 to produce the drive force to repeatably move both the plungers 2320 of both the first container 2301 and the second container 2302. In other words, due to the coupling of the plungers 2320 via the plunger link 2370, the plunger 2320 of the first container 2301 and the plunger 2320 of the second container 2302 move distally in unison in response to an input from the delivery device 2100. However, in some embodiments, the contact face 2167 of the second drive member portion 2164 is a non-planar contact face and the plungers 2320 are decoupled from the plunger link. For example, referring to FIG. 19, in some embodiments a second drive member 2192 can include a non-planar contact face 2167. Accordingly, the elastomeric member 2315 of the first container 2301 and the elastomeric member 2315 of the second container 2302 travel a different distance in response to a single stroke length of the motive assembly 2150. Such a configuration facilitates alternative mixing ratios of the first and second component and/or the utilization of containers of dissimilar dimensions.

As shown, the connector 2400 includes a first (or input) end portion 2401 and a second (or output) end portion 2402. The first end portion 2401 is configured to receive a distal end (or connector) of the first container 2301 and a distal end (or connector) of the second container 2302. The second end portion 2402 is configured to be coupled to a delivery member (see e.g., FIG. 1). In this manner, the first component can be conveyed from the first container 2301, into the first end portion 2401 of the connector 2400, and out of the second end portion 2402 of the connector to the delivery member. Similarly, the second component can be conveyed from the second container 2302, into the first end portion 2401 of the connector 2400, and out of the second end portion 2402 of the connector 2400 to the delivery member. In some embodiments, the connector 2400 can be a mixing connector within which the first component is mixed with the second component before the two components are conveyed into the delivery member. In other embodiments, however, the connector 2400 can maintain the first component separate from the second component, and the two components are conveyed into and mixed within the delivery member. By maintaining separate flow paths within the connector 2400, the reaction (e.g., crosslinking) between the first component and the second component can be performed outside of the connector 2400 (i.e., within the delivery member), thereby limiting the likelihood of clogging with the connector 2400. In this manner, the connector 2400 can be used for multiple injections.

In use, after the container 2300 is prepared, assembled into the cartridge assembly 2500, and coupled to the delivery device 2100, the motive assembly 2150 can be actuated to produce the drive force. In this manner, the motive assembly 2150 (and the drive member 2160) can move the plungers of the first and second containers 2301, 2302 simultaneously for a time period to dispense a portion of the first component from the first container 2301 and a portion of the second component from the second container 2302. The first component and the second component are conveyed through the connector 2400 and to the target location via a delivery member (not shown). As described above, the first component and the second component can react (e.g., within the delivery member) to form the biomaterial product.

In some embodiments, the delivery system 2000 can be used to perform multiple depositions using the same container 2300. For example, in some embodiments, the delivery system 2000 (and any of the systems described herein) can be used to deliver a biomaterial product (e.g., a hydrogel) to occlude each of the two vas deferens of a patient. In such embodiments, the container 2300 can include a sufficient amount of the first component and the second component to deliver two separate biomaterial products to the patient (one for each vas deferens). In such embodiments, the delivery system 2000 can be primed two times (once before each of the injection events).

The container 2300 (and any of the containers described herein, such as the first container 2301 and/or the second container 2302) can be filled with the first component prior to being coupled to the cartridge assembly 2500 and/or the delivery device 2100. The container 2300 can be prefilled with the first component or alternatively the user can mix and/or prepare the first component on site and then manually fill the container 2300 before use. The amount of the first component within the container 2300 will vary between a maximum fill volume and a minimum fill volume. In use, upon initiating the first prime event, the elastomeric member 2315 will move through a first prime distance from a starting position (i.e., a home position of the drive member 2160 and/or the motive assembly 2150) to a first prime position. During the first prime event, the first component and the second component will be conveyed from the first container 2301 and the second container 2302, respectively, and the connector 2400. In some embodiments, because the first component is separate from the second component within the connector 2400, no crosslinking of the components takes place during the first prime operation. The user can wipe the residual material from the tip of the connector 2400. The delivery system 2000 is then ready to be coupled to the delivery member to complete the first injection (e.g., deposition). This operation may be followed by a subsequent operation.

As described above, the drive member 2160 and/or the motive assembly 2150 is associated with a starting (or home) position, which is the position from which the drive member 2160 initially begins a sequence of operations and/or to which the drive member 2160 returns after completing a sequence of operations. The home position can be maintained at a constant (or substantially constant) distance from the flange 2314 of the container 2300 (and any of the containers described herein). By maintaining a constant home position relative to the container 2300, the delivery device 2100 can repeatably deliver the desired amounts of the components for multiple different operations, with the same or different patients, using multiple different containers 2300 (e.g., different container assemblies).

In some embodiments, the motive assembly 2150 and/or the delivery device 2100 can include an electronic control system 2200 that controls the electromechanical driver 2152 and any other aspect of the motive assembly 2150 to control the delivery characteristics of the composition and/or the delivered product, as described herein. The electronic control system 2200 is coupled to an energy storage member 2240 (e.g., FIG. 18) and includes one or more sensors, one or more processors, one or more memory components, and various modules, such as a drive module and a user interface module. Although FIG. 18 illustrates the electronic control system 2200 being within the housing 2110, the electronic control system 2200 or portions thereof can be provided outside of the housing 2110 (e.g., certain operations of the electronic control system 2200 can be performed within a cloud computing environment). As described herein, the electronic control system 2200 can automatically control the duration of delivery, the velocity of the components, the peak force applied during delivery, and any other aspects of delivering the composition.

Specifically, the electronic control system 2200 can control the motive assembly 2150 based on feedback from the sensor(s). The sensor(s) can be separate and/or included within the electronic control system 2200 can include any suitable sensor that produces a feedback signal associated with at least one of a position of the drive member 2160, a velocity of the drive member 2160, an acceleration of the drive member 2160, or the drive force applied by the drive member 2160. Such sensors can include, for example, imaging devices, optical sensors, accelerometers, temperature sensors, contact sensors, proximity sensors, position sensors, and/or any other suitable input device. For example, in some embodiments, the sensor can be a linear position sensor (e.g., an LVDT or the like) that produces a feedback signal associated with the position of the drive member 2160 or a component of the motive assembly 2150. The feedback signal can also be used to determine changes in position during the delivery event (e.g., the velocity of the drive member 2160). For example, the sensor can be a rotational sensor that monitors the rotations of the electromechanical driver 2152 and/or a lead screw coupled thereto over a specified time interval. In other embodiments, the sensor can be a force sensor (e.g., a strain gauge force sensor) that produces a feedback signal associated with the force applied by or exerted on the drive member 2160. In yet other embodiments, the sensor can be a current sensor that measures the current and/or voltage supplied to the electromechanical driver. From the measured current and/or voltage, the electronic control system 2200 (e.g., the drive module) can calculate the power, and therefore, the approximate force applied by the drive member 2160. The sensor can also be other type of sensor, such as an accelerometer (to measure vibration, motion and/or acceleration of the drive member 2160), an optical sensor(s) to detect certain positions of the drive member 2160 and/or cartridge assembly 2500, or one or more switches. Although described as including one feedback sensor, the electronic control system 2200 (and any of the electronic control systems described herein) can include any number of sensors. For example, in some embodiments, the electronic control system 2200 can include a linear position sensor to measure the position of the drive member 2160 and an optical sensor to determine whether a connector 2400 is coupled to the container 2300. In some embodiments, the electronic control system 2200 is configured to monitor a position the plunger 2320 relative to the drive member 2160 and/or the housing 2110 to improve accuracy and control of the plunger 2320 during a priming operation and/or a deposition operation.

In some embodiments, the electronic control system 2200 is configured to detect and verify that the cartridge assembly 2500 and/or the connector assembly 2400 are properly mounted and coupled to the housing 2110. Accordingly, the electronic control system 2200 can include a positional sensor 2250 (e.g., FIG. 18) coupled to the housing 2110. The positional sensor 2250 can be a photoelectric sensor, an ultrasonic position sensor, an inductive proximity sensor, an electrical contact sensor, a magnetic contact sensor, a reflective laser sensor, a compression sensor, or other similar sensor. The positional sensor 2250 can be operatively engaged by the cartridge chassis 2510 only when the container chassis is in a nominal longitudinal position and/or lateral position. For example, a portion of the cartridge chassis 2510 can be sized to engage the position sensor 2250 by occluding a photoelectric sensor only when the cartridge chassis 2510 is in the nominal longitudinal position. This arrangement allows the positional sensor 2250 to produce a signal indicating either proper alignment or misalignment of the cartridge chassis 2510. The sensor interface feature 2522 can any suitable feature that can operably engage with the sensor, such as, for example, an opening, a transparent region of the cartridge chassis 2510, a magnetic portion, a reflective region of the cartridge chassis 2510, or a colored region of the cartridge chassis.

The processor, and any of the processors described herein can be any suitable processor for performing the methods described herein. In some embodiments, processor can be configured to run and/or execute application modules, processes and/or functions associated with the delivery device 2100. For example, the processor can be configured to run and/or execute the drive module, the user interface module (which functions an input/output module), and/or any of the other modules described herein, and perform the methods associated therewith. The processor can be, for example, a Field Programmable Gate Array (FPGA), an Application Specific Integrated Circuit (ASIC), a Digital Signal Processor (DSP), and/or the like. The processor can be configured to retrieve data from and/or write data to memory, e.g., the memory.

The memory (or any of the memory devices described herein) can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth. In some embodiments, the memory stores instructions to cause the processor to execute modules, processes and/or functions associated with the delivery device 2100. For example, the memory can store instructions to cause the processor to execute any of the application modules described herein, and perform the methods associated therewith.

The user interface module can be a hardware and/or software module (stored in memory and/or executed in the processor). The user interface module can be configured to receive input from and/or produce output to the user. For example, the user can depress the actuator button 2230, which is operatively coupled to the electronic control system 2200. The user interface module can receive the input and produce one or more signals based on the user input. For example, in some embodiments, the user interface module can produce a signal to the motive assembly 2150 to “prime” the system by moving the drive member 2160 a predetermined distance. In other embodiments, the user interface module can produce a signal to the motive assembly 2150 to stop movement of the drive member 2160 (e.g., in an error state or “stop” state). The user interface module can also produce a signal to cause the electronic control system 2200 to produce one or more outputs. For example, in some embodiments, the electronic control system 2200 (and any of the electronic control systems described herein) includes an output device 2221, such a light output device (e.g., LED's), an audible output device (e.g., speaker), or a tactile output device (e.g., vibration device). In such embodiments, the user interface module can produce a signal to cause the output device 2221 to produce an output (e.g., a visual, audible, or tactile output) to indicate a change in state (e.g., priming completed, mixing completed, injection complete) or error condition associated with the delivery device 2100. In some embodiments, the user interface module includes a graphical user interface to display information relating to the delivery system 2000 and to receive inputs from a user.

The drive module can be a hardware and/or software module (stored in memory and/or executed in the processor). The drive module can be configured to receive the feedback sensor (e.g., from the sensor(s)) and produce, based on the feedback signal, a drive signal to maintain the drive force below a drive force threshold. For example, after the container 2300 is prepared and coupled to the delivery device 2100, the motive assembly 2150 can be actuated (e.g., via the actuator button 2230) to produce the drive force. In this manner, the motive assembly 2150 (and the drive member 2160) can move the plunger 2320 for a time period to dispense a portion of the composition from the container 2300. The drive module is configured produce a drive signal to maintain the drive force below a drive force threshold during the delivery time period. The drive signal can be, for example, a control signal that adjusts a level of power (e.g., current or power) applied to the motive assembly 2150 to maintain the drive force below the drive force threshold. In some embodiments, the motive assembly 2150 can include an electromechanical driver 2152, such as a stepper motor. The drive signal can be a series of pulses to maintain and/or control the speed of the motor, thereby maintaining the force below the drive force threshold.

In some embodiments, the drive module can be configured to maintain the drive force below more than one threshold. For example, in some embodiments, the injection can be divided into two intervals. During a first interval, which can be referred to as a “breakaway” period, the drive module can be configured to maintain the drive force below a breakaway force threshold. During a second interval, which can be referred to as a “delivery” period, the drive module can be configured to maintain the drive force below a drive force threshold. The drive force threshold may be lower than the breakaway force threshold. By controlling motive assembly 2150 to produce a higher force on the “startup” period (i.e., the period during which movement of the plunger 2320 is initiated), the system can overcome static friction, while still minimizing the high force impulse to limit damage at the target location (e.g., tissue damage). In other embodiments, the drive force threshold can be equal to or higher than the breakaway force threshold. In yet other embodiments, the drive force can be maintained below more than two different thresholds. For example, in some embodiments, the drive module can be configured to maintain the drive force below a drive threshold force curve that changes as a function of time. In some embodiments, the drive threshold curve can remain constant, increase, and/or decrease as a function of time.

In some embodiments, the drive module and/or the user interface module is configured to produce an error signal when the drive force exceeds the drive force threshold. In some embodiments, the error signal can cause the output device 2221 to produce any one of a visual, audible, or tactile output. In other embodiments, the error signal can cause a reduction in the power or drive signal to the motive assembly 2150. Similarly stated, the error signal can cause the motive assembly 2150 to reduce and/or stop movement to limit the likelihood of damage that target location caused by excessive force. In yet other embodiments, the drive module can produce a completion signal at the end of the delivery event to cause the motive assembly 2150 to stop movement.

In embodiments wherein the composition is a biomaterial that is implanted in a patient, maintaining the drive force below the drive force threshold, facilitates delivery of the biomaterial in a manner that limits the likelihood of tissue damage (e.g., due to excessive velocity and/or force causing potential tissue damage). Specifically, the drive force (i.e., the force applied by the delivery member) is related to the force with which the biomaterial is delivered to the target location (i.e., the delivery force). For example, the drive force generates a pressure within the container assembly that moves the elastomeric member thereby causing the flow composition through the system. The drive force is counteracted by friction forces within the system as wells as pressure from the delivery site. Thus, the delivery force, which is applied to and/or within the target tissue, is associated with the drive force and the losses (e.g., friction forces) through the system. The drive force threshold can be any suitable value. For example, in some embodiments, the drive force can be maintained between 0.1N and about 45N. In other embodiments, the drive force can be maintained below about 20N. In yet other embodiments, the drive force can be maintained below about 12N and/or within a range of between about 7N and 12N.

In some embodiments, the drive module can be configured to maintain a velocity of the components exiting the delivery system 2000 within a desired velocity range, similar to the ranges described above with respect to the delivery device 2100. As described above, the drive force needed to deliver the components from the container 2300 and the composition exiting a delivery member is related to the target flow rate (and therefore the velocity), the properties of the materials (e.g., viscosity), and the characteristics of the delivery system 2000 (e.g., length of the delivery member and friction between the elastomeric member 2315 and the container 2300). In certain circumstances (i.e., laminar flow of the components), the pressure of the composition within the container 2300 (which is related to the drive force exerted by the motive assembly 2150) can be modeled by the Hagen-Poiseuille law, as indicated below:

$$P = ( 8 * μ * L * Q ) / ( Π * {R}^{4} ) \qquad ( 1 )$$

where P is the pressure of the composition within the container 2300, u is the viscosity of the composition, L is the length of the delivery member, Q is the flow rate of the composition through the delivery member, and R is the radius of the lumen defined by the delivery member. Because the pressure (and therefore drive force) required to inject a high viscosity fluid through a small-diameter delivery member is proportional to the inverse of the radius of the lumen of the delivery member to the fourth power, the pressure of the components within the container 2300 must be high enough to achieve the desired flow rate, while being controlled to avoid excess flow rates (or velocities) and/or high delivery (or exit) forces, which could damage the target tissue. Furthermore, for certain applications, it is necessary to control the injection location, rate of delivery, and volume of the biomaterial delivered such that implantation of the biomaterial into that tissue space does cause the material to enter or damage other tissue spaces. For example, if the injection is too slow based upon the mechanical properties and pressures at the implantation site this can cause a poorly formed implant or leakage from entry site for implantation or clogs. For example, if the injection is too fast based upon the mechanical properties and pressures at the implantation site this can cause excessive tissue damage or vessel rupture or foreign body response from damage of implantation.

As depicted in FIGS. 2-6, 8-12, 18, and 19, the housing 2110 defines a coupling interface 2170 that is configured to receive the cartridge chassis 2510. The coupling interface 2170 includes at least one longitudinal positioning feature 2172. The longitudinal positioning feature(s) 2172 is configured to engage a corresponding longitudinal positioning feature 2560 (e.g., a positioning recess) of the cartridge chassis 2510 and establish a longitudinal position of the cartridge chassis 2510. Similarly stated, the longitudinal positioning feature(s) 2172 and the corresponding longitudinal positioning feature(s) 2560 of the cartridge chassis 2510 are configured to secure the chassis 2510 at a reference position relative to the housing 2110 and to limit (or prevent) movement of the chassis 2510 relative to the housing 2110. The housing 2110 also defines a mounting surface 2174 of the coupling interface 2170 that has a maximal dimension that extends between the distal direction (D) and the proximal direction (P).

The cartridge chassis 2510 includes a plurality of coupling members 2550. For example, as depicted in FIGS. 2, 3, 6, 8, and 13-15, the cartridge chassis 2510 can include four coupling members 2550 to establish four points of securement with the housing 2110. However, in some embodiments the cartridge chassis 2510 can include two coupling members 2550, three coupling members 2550, or more than four coupling members 2550. The plurality of coupling members 2550 are distributed about a perimeter 2502 of the cartridge chassis 2510. Each coupling member of the plurality of coupling members 2550 is positioned to be received by the coupling interface 2170 of a delivery device 2100. As depicted, in some embodiments, the coupling interface 2170 receives the plurality of coupling members 2550 in a side-side configuration. However, in additional embodiments, the coupling interface 2170 receives the plurality of coupling members 2550 in a proximal-distal configuration. (such as depicted in FIG. 1).

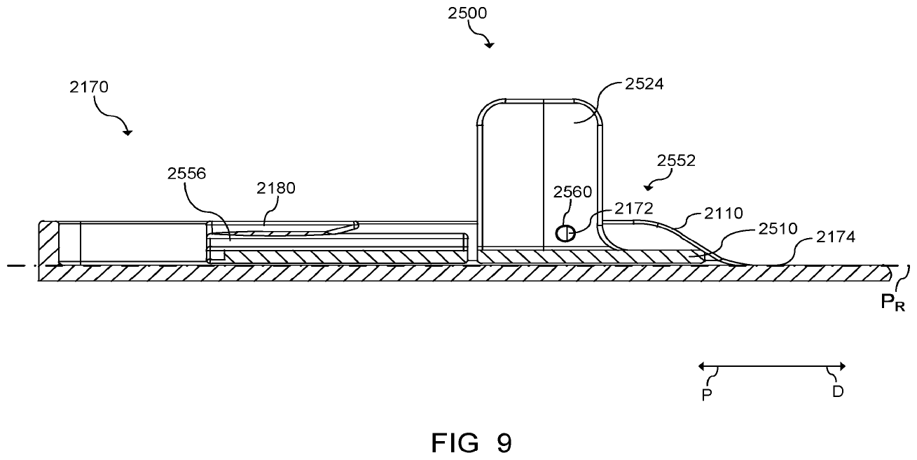

At least one coupling member 2550 includes a longitudinal positioning feature 2560 positioned to engage a first corresponding feature (e.g. longitudinal positioning feature 2172) of the coupling interface 2170 of the delivery device 2100. For example, the longitudinal positioning feature 2560 of the coupling member 2550 can be a recess, a depression, or a through-hole sized to receive the longitudinal positioning feature 2172 of the coupling interface 2170 when configured as a protrusion as depicted in FIGS. 9-11, and 19. However, in additional embodiments, the longitudinal positioning feature 2560 of the coupling member 2550 may be formed as a protrusion positioned to be received by a corresponding recess, depression, or through-hole defined by the coupling interface 2170 (e.g., by the housing 2110). In some embodiments, the corresponding longitudinal positioning features 2560, 2172 may be formed with complementary tapering side portions thereby facilitating the centering of the protruding portion within the receiving portion.

The engagement of the corresponding longitudinal positioning features 2560, 2172 prevents longitudinal movement of the cartridge chassis 2510 relative to the coupling interface 2170. Additionally, the cartridge chassis 2510 includes a container clip 2512 that, at least partially, surrounds the container body 2310 and secures the container 2300 in a fixed position relative to the cartridge chassis 2510. Accordingly, when the cartridge assembly 2500 is coupled to the delivery device 2100, the container 2300 is established in a fixed position relative to the delivery device 2100. In other words, the container 2300 is fixed in a first coordinate system (x₁, y₁, z₁) (FIG. 13) defined by the cartridge chassis 2510. The cartridge chassis 2510 is fixed in a second coordinate system (x₂, y₂, z₂) (FIG. 2) defined by the delivery device 2100.

At least one coupling member 2550 includes a living hinge 2562. The living hinge 2562 is oriented to exert a force on the delivery device 2100 (e.g., on the housing 2110) when the cartridge assembly 2500 is received by the coupling interface 2170. For example, as depicted, each living hinge 2562 of the plurality of coupling members 2550 is oriented to exert a lateral force on the housing 2110. Accordingly, the coupling member(s) 2550 has an extended position or a neutral position when the cartridge chassis 2510 is separated from the housing 2110 of the delivery device 2100 and a compressed position when received by the coupling interface 2170. In some embodiments, the engagement between at least one coupling member 2550 and the coupling interface 2170 also develops a force that is substantially orthogonal to the mounting surface 2174 (e.g., in the z₂ direction). In this manner, the cartridge chassis 2510 can be secured to the mounting surface 2174 and movement away from the mounting surface (i.e., in a direction substantially orthogonal to the mounting surface 2174) can be limited or prevented. As described above, by limiting (or preventing) movement of the cartridge chassis 2510 relative to the housing 2110 during use, the accuracy of the delivery of materials from within the container 2300 can be improved.

In some embodiments, the plurality of coupling members 2550 are each monolithically formed with the cartridge chassis 2510. However, in additional embodiments, at least one coupling member the plurality of coupling members 2550 is coupled to the cartridge chassis 2510.

As depicted, the plurality of coupling members 2550 includes a first coupling member 2552. The first coupling member 2552 is formed at a distal perimeter portion 2503 of the cartridge chassis 2510. The first coupling member 2552 includes a living hinge 2562. The living hinge 2562 of the first coupling member 2552 has an extended position when the cartridge chassis is separated from the delivery device 2100. The living hinge 2562 has a compressed position when received by the coupling interface 2170. The living hinge is oriented to exert a force (e.g., a first force) on the delivery device 2100 when received by the coupling interface 2170. In other words, in the compressed position, the living hinge 2562 exert a lateral force on the housing 2110. The force exerted by the living hinge 2562 may facilitate the retention of the longitudinal positioning feature 2172 of the coupling interface 2170 by the longitudinal positioning feature 2560 of the first coupling member 2552.

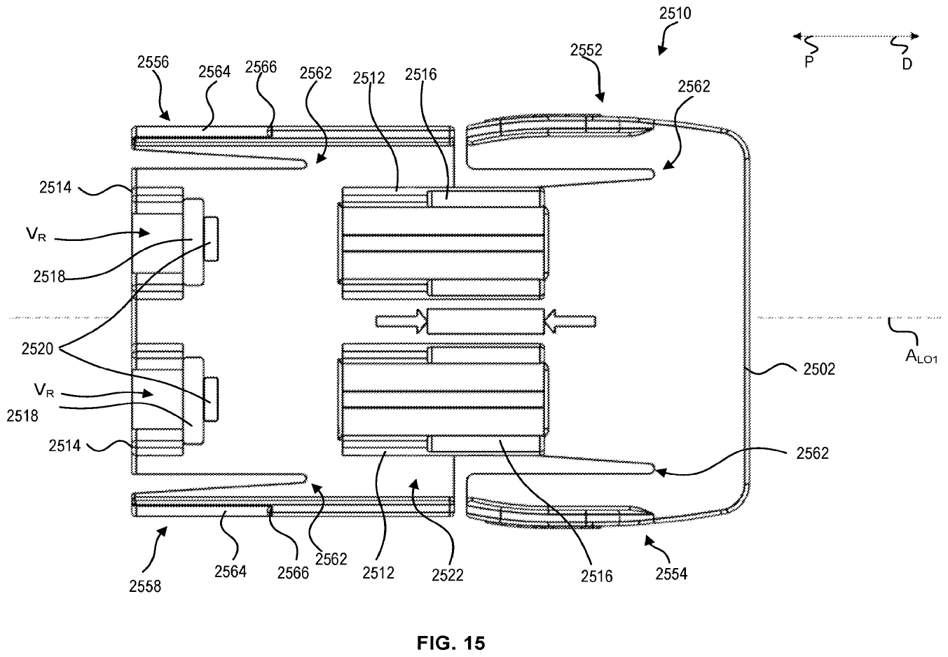

In some embodiments, the distal perimeter portion 2503 of the cartridge chassis 2510 includes a second coupling member 2554 of the plurality of coupling members 2550. The second coupling member 2554 is positioned opposite the first coupling member 2552 about a longitudinal axis (A_{LO1}) (FIG. 15) of the cartridge chassis 2510. The second coupling member 2554 includes a living hinge 2562. The living hinge 2562 of the second coupling member 2554 has an extended position when the cartridge chassis is separated from the delivery device 2100. The living hinge 2562 has a compressed position when received by the coupling interface 2170. The living hinge is oriented to exert a force (e.g., a second force) on the delivery device 2100 when received by the coupling interface 2170. In other words, in the compressed position, the living hinge 2562 exert a lateral force on the housing 2110. The force exerted by the living hinge 2562 may facilitate the retention of the longitudinal positioning feature 2172 of the coupling interface 2170 by the longitudinal positioning feature 2560 of the second coupling member 2554. In additional embodiments, however, the second coupling member 2554 can be formed as a fixed feature, such as a protrusion, lip, tab, or edge that is received by a portion of the coupling interface 2170.

As depicted, a proximal perimeter portion 2504 of the cartridge chassis 2510 can include a third coupling member 2556 of the plurality of coupling members 2550. The third coupling member 2556 includes a living hinge 2562 (e.g., a third living hinge) that is defined by the proximal perimeter portion 2504. The living hinge 2562 of the third coupling member 2556 has a neutral position when the cartridge chassis 2510 is separated from the delivery device 2100. The living hinge 2562 has a compressed position when received by the coupling interface 2170. The living hinge is oriented to exert a force (e.g., a third force) on the delivery device 2100 when received by the coupling interface 2170. The living hinge 2562 is oriented to exert a force on the delivery device 2100 when received by the coupling interface 2170. In other words, in the compressed position, the living hinge 2562 of the third coupling member 2556 exerts at least a lateral force on the housing 2110 when the cartridge assembly 2500 is coupled to the delivery device 2100.

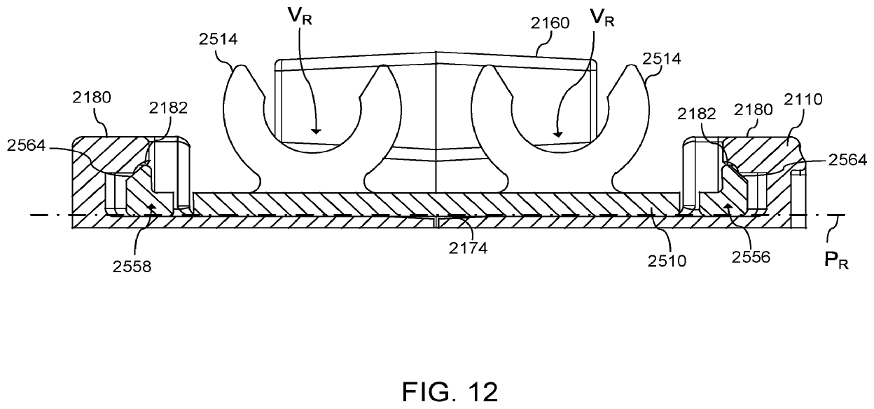

As depicted in FIGS. 12-16, the third coupling member 2556 of the cartridge chassis 2510 includes a contact face 2564 (e.g., a third contact face). The contact face 2564 is configured to be engaged between the mounting surface 2174 of the coupling interface 2170 and a proximal retaining portion 2180 of the coupling interface 2170. In other words, a portion of the third coupling member 2556 is positioned (e.g., inserted) between the mounting surface 2174 of the housing and a protrusion of the housing 2110 as depicted in the cross-sectional view of FIG. 9. In some embodiments, the third coupling member 2556 of the cartridge chassis 2510 includes a hard-stop 2566. The hard-stop 2566 is positioned to engage the proximal retaining portion 2180 of the housing 2110 and limit longitudinal movement of the cartridge chassis 2510 in the proximal direction (e.g. along the longitudinal axis (A_{LO1})). The hard-stop 2566, in some embodiments, is positioned abutting a distal most portion of the contact face 2564. As such, the hard-stop 2566 can establish a maximal proximal position of the cartridge chassis 2510 relative to the delivery device 2100.

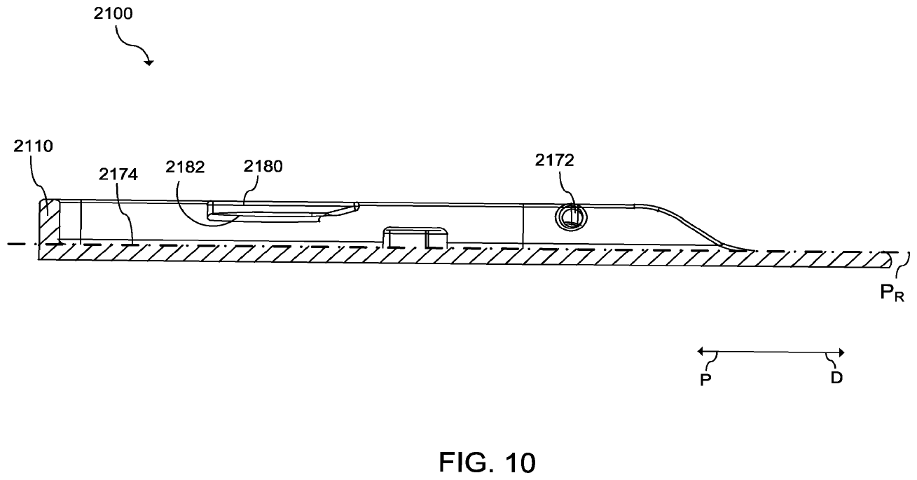

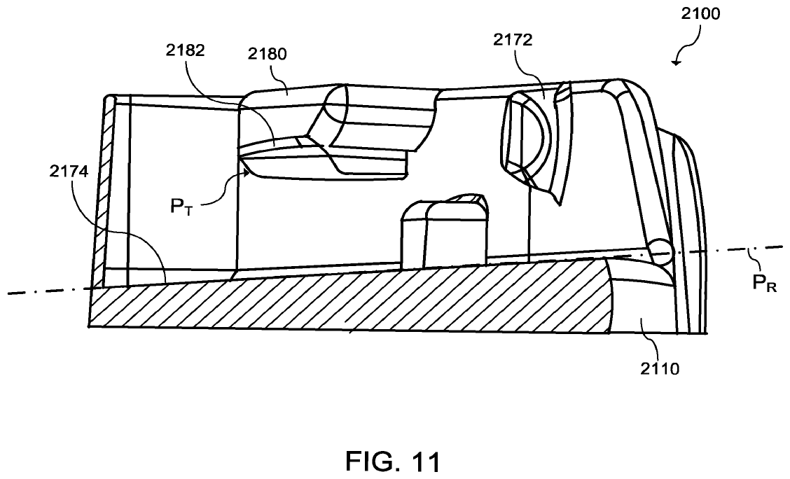

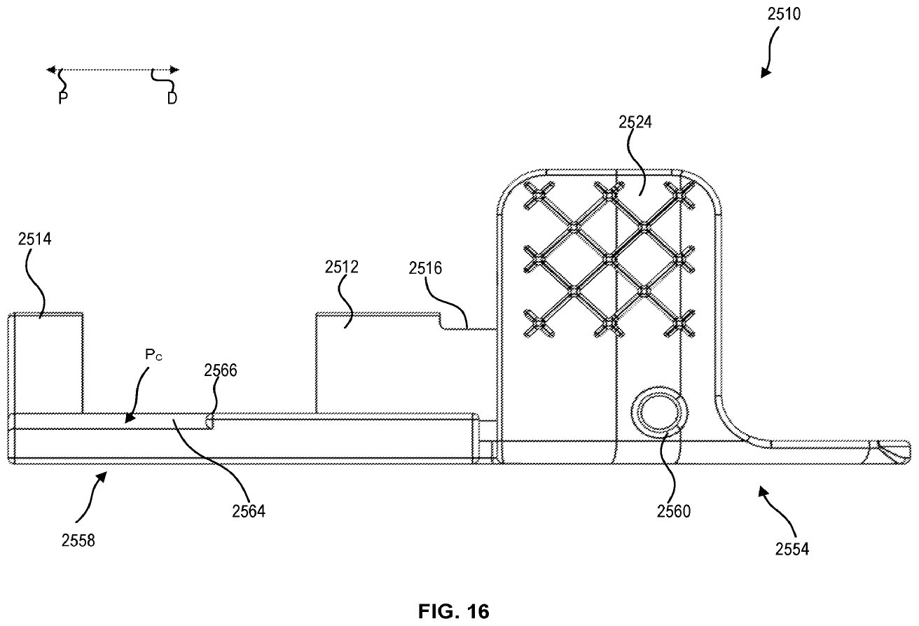

The contact face 2564 of the third coupling member 2556 defines a contact plane (Pc) (e.g., a first contact plane (Pc)) as depicted in FIGS. 13 and 16. The contact plane (Pc) extends in the longitudinal direction (e.g., along the longitudinal axis (A_{LO1})) relative to the cartridge chassis 2510. As illustrated in FIGS. 10-12, the proximal retaining portion 2180 of the housing 2110 includes a retention face 2182. The retention face 2182 of the coupling interface 2170 is oriented toward (e.g., facing) the mounting surface 2174 of the coupling interface 2170 (e.g., of the housing 2110). As depicted in FIG. 12, the retention face 2182 is positioned to oppose the contact face 2564 when the cartridge assembly 2500 is coupled to the delivery device 2100. As depicted in FIG. 11, the retention face 2182 defines a retention plane (PT). The retention plane (PT) is oriented at a compound angle relative to the mounting surface 2174. Accordingly, the engagement of the contact face 2564 of the third coupling member 2556 of the cartridge chassis 2510 and the retention face 2182 of the proximal retaining portion 2180 of the coupling interface 2170 develops a first force that is parallel to the mounting surface 2174 and a second force that is orthogonal to the mounting surface 2174. Said another way, the intersection of the retention plane (PT) and the contact plane (Pc), in combination with the force generated by the living hinge 2562, results in a first force that fixes the position of the cartridge chassis 2510 along the axis y₂ (e.g., laterally) and a second force that fixes the position of the cartridge chassis 2510 along the axis z₂ (e.g., vertically). In this manner, the cartridge chassis 2510 can be secured to the mounting surface 2174 and movement away from the mounting surface (i.e., in a direction substantially orthogonal to the mounting surface 2174) can be limited or prevented. As described above, by limiting (or preventing) movement of the cartridge chassis 2510 relative to the delivery device 2100 during use, the accuracy of the delivery of materials from within the container 2300 can be improved.

In some embodiments the proximal perimeter portion 2504 of the cartridge chassis 2510 includes a fourth coupling member 2558 of the plurality of coupling members 2550. The fourth coupling member 2558 is positioned opposite the third coupling member 2556 about the longitudinal axis (A_{LO1}) of the cartridge chassis 2510. In some embodiments, the fourth coupling member 2558 can include a living hinge 2562 (e.g., a fourth living hinge) and a contact face 2564 (e.g., a fourth contact face) engaged between the mounting surface 2174 and the proximal retaining portion 2180 as described above. Accordingly, the fourth coupling member 2558 generate the forces described above with reference to the third coupling member 2556. However, in additional embodiments the fourth coupling member 2558 can be formed as a fixed feature, such as a protrusion, lip, tab, or edge that is received between the mounting surface 2174 and the proximal retaining portion 2180.

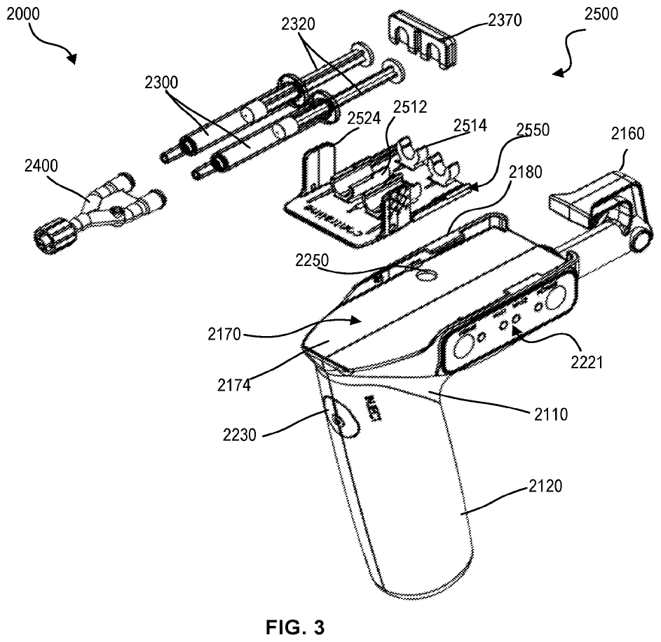

As previously described, the cartridge chassis 2510 includes at least one container clip 2512 that at least partially surrounds the container body 2310 to secure the container 2300 in a fixed position relative to the cartridge chassis 2510. As depicted in FIGS. 3 and 12-15, in some embodiments, the cartridge chassis 2510 includes a plunger guide structure 2514. The plunger guide structure 2514 is in axial alignment with the container clip 2512. The plunger guide structure 2514 is positioned to at least partially circumscribe the plunger 2320 of the container 2300. When the container 2300 is secured by the container clip 2512, the flange 2314 of the container 2300 abuts the plunger guide structure 2514. Accordingly, the plunger guide structure 2514 limits a longitudinal movement of the container 2300 in the proximal (P) direction.

The plunger guide structure 2514 defines a receiving volume (VR). The receiving volume (VR) has a radius that is less than a radius of a receiving volume (VR) defined by the container clip 2512. In some embodiments, the radius of the receiving volume (VR) of the plunger guide structure 2514 is less than the radius of the container body 2310. The radius differences between the plunger guide structure 2514 and the container clip 2512 preclude coupling the container 2300 to the cartridge chassis 2510 in a reversed orientation (e.g., in an orientation with the plunger 2320 toward the distal end of the cartridge chassis 2510).

In some embodiments, the container clip 2512 defines a nominal positioning indicator 2516. The nominal positioning indicator 2516 corresponds to a nominal position of a distal end 2316 of the elastomeric member 2315 when the container 2300 is secured by the container clip 2512. The nominal positioning indicator 2516 provides an indication that the container 2300 is properly positioned relative to the cartridge chassis 2510 and contains a nominal volume of the composition. The nominal position indicator 2516 may be a colored and/or graduated region of the container clip 2512. The nominal position indicator 2516 may indicate a nominal position point or may indicate a nominal position range.

As depicted in FIG. 15, the cartridge chassis 2510 defines at least one recess 2518. The recess 2518 is positioned between the plunger guide structure 2514 and the container clip 2512. The recess 2518 is aligned with the plunger guide structure 2514 and the container clip 2512. The recess 2518 has a long edge oriented orthogonal to the longitudinal axis (A_{LO1}) of the cartridge chassis 2510. The recess 2518 is sized to receive a portion of the flange 2314 of the container 2300. As depicted in FIG. 17, the recess 2518 is sized such that a longitudinal axis (A_{LO2}) of the container body 2310 is parallel to the mounting surface 2174 of the coupling interface 2170 when the portion of the flange 2314 is within the recess 2518 and when the cartridge chassis 2510 is received by the coupling interface 2170. In other words, the size of the recess 2518 is tailored to the container 2300. For example, an oversized recess 2518 may accept too great a portion of the flange 2314 and thereby permit a proximal portion of the container body 2310 to be closer to the mounting surface 2174 than a distal portion of the container body 2310. The converse is true for an undersized recess 2518.

As depicted in FIGS. 16 and 17, the cartridge chassis 2510 includes a stop protrusion 2520. The stop protrusion 2520 extends from the cartridge chassis 2510 along the axis z₁ (e.g., vertically). The stop protrusion 2520 is positioned between the recess 2518 and the container clip 2512. In this position, the stop protrusion 2520 engages the flange 2314 of the container 2300. The engagement of the flange 2314 by the stop protrusion 2520 preclude the movement of the container body 2310 in a distal direction (D). Said another way, the flange 2314 is sandwiched in the recess 2518 and between the stop protrusion 2520 and the plunger guide structure 2514. This arrangement precludes the movement of the container body 2310 in both the proximal (P) direction and the distal (D) direction.

The cartridge chassis 2510 includes a sensor interface feature 2522. The sensor interface feature 2522 is configured to operably engage with the positional sensor 2250 (FIG. 18) of the delivery device 2100. The positional sensor 2250 can be a photoelectric sensor, an ultrasonic position sensor, an inductive proximity sensor, an electrical contact sensor, a magnetic contact sensor, a reflective laser sensor, a compression sensor, or other similar sensor. In some embodiments, the sensor interface feature 2522 can be sized to preclude the engagement of the positional sensor for a longitudinal position of the cartridge chassis 2510 that deviates from a nominal longitudinal position. For example, the sensor interface feature 2522 can be sized to engage the position sensor by occluding a photoelectric sensor only when the cartridge chassis 2510 is in the nominal longitudinal position.

In some embodiments, the cartridge chassis 2510 includes a manual engagement structure 2524. The manual engagement structure 2524 is positioned to facilitate removal of the cartridge chassis 2510 from the delivery device 2100. The manual engagement structure 2524 can be monolithically formed with the cartridge chassis 2510 or coupled thereto. For example, in some embodiments, the cartridge chassis 2510 includes a pair of manual engagement structures 2524 is positioned such that a force applied to the pair of manual engagement structures 2524 generates a bending moment about the longitudinal axis (A_{LO1}). The resultant bending of the cartridge chassis 2510 generate a displacement of the first and/or second coupling members 2552, 2554 toward the longitudinal axis (A_{LO1}). The displacement of the first and/or second coupling members 2552, 2554 decouples the cartridge chassis 2510 from the longitudinal positioning feature(s) 2172 of the coupling interface 2170 and, thus, the delivery device 2100.

In some embodiments, the cartridge assembly 2500 (and any cartridge assembly described herein) is delivered to an operator of the delivery system 2000 as a limited-dosage kit. For example, in some embodiments, the limited-dosage kit contains the cartridge chassis 2510, the first container 2301, the second container 2302, the coupler 2400, the plunger link 2370, the delivery member, and constituents of the first and second components of the composition. Accordingly, the first and second components of the composition may be prepared and introduced into the first container 2301 and the second container 2302 respectively. The first and second container 2301, 2302 are then coupled to the coupler 2400 and the plunger link 2370 (if any) is affixed to the plungers 2320. The first and second containers 2301, 2302 are then coupled to the cartridge chassis 2510 and the cartridge chassis 2510 is coupled to the delivery device 2100. With the first and second containers 2301, 2302 and fixed longitudinal positions relative to the delivery device 2100, the operator of the delivery system 2000 verifies that the distal end 2316 of the elastomeric member 2315 is in the nominal position as indicated by the nominal positioning indicator 2516. Following the verification of the nominal position, the delivery system 2000 is employed to deliver a dosage of the composition to the target location.

A number of embodiments have been described. Nevertheless, it will be understood that various modifications may be made without departing from the spirit and scope of the invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Any of the components and sub-components described herein can be included in any of the embodiments unless mutually exclusive.

1. (canceled)

2. A cartridge assembly, comprising:
a container including a container body and an elastomeric member disposed within the container body to retain a composition within the container;
a cartridge chassis having a plurality of coupling members distributed about a perimeter of the cartridge chassis, each coupling member of the plurality of coupling members is positioned to be received by a coupling interface of a delivery device, the cartridge chassis including a container clip at least partially surrounding the container body to secure the container in a fixed position relative to the cartridge chassis, a first coupling member of the plurality of coupling members including a longitudinal positioning feature positioned to engage a first corresponding feature of the coupling interface of the delivery device to prevent longitudinal movement of the cartridge chassis relative to the coupling interface; and
the first coupling member is formed at a distal perimeter portion of the cartridge chassis and includes a living hinge, the first coupling member having an extended position when the cartridge chassis is separated from the delivery device and a compressed position when received by the coupling interface, the living hinge being oriented to exert a force on the delivery device when received by the coupling interface.

3. The cartridge assembly of claim 2, wherein:
the distal perimeter portion of the cartridge chassis includes a second coupling member of the plurality of coupling members, the second coupling member being positioned opposite the first coupling member about a longitudinal axis of the cartridge chassis.

4. The cartridge assembly of claim 3, wherein:
the living hinge is a first living hinge;
the force is a first force; and
the second coupling member includes a second living hinge and has an extended position when the cartridge chassis is separated from the delivery device and a compressed position when received by the coupling interface, the second coupling member including a second longitudinal positioning feature with the second living hinge being oriented to exert a second force on the delivery device when received by the coupling interface.

5. The cartridge assembly of claim 2, wherein:
a proximal perimeter portion of the cartridge chassis includes a third coupling member of the plurality of coupling members, the third coupling member including:
a living hinge defined by the proximal perimeter portion and having a neutral position when the cartridge chassis is separated from the delivery device and a compressed position when received by the coupling interface, the living hinge being oriented to exert a force on the delivery device when received by the coupling interface, and
a contact face configured to be engaged between a mounting surface of the coupling interface and a proximal retaining portion of the coupling interface of the delivery device.

6. The cartridge assembly of claim 5, wherein:
the contact face defines a contact plane extending in the longitudinal direction; and
the proximal retaining portion includes a retention face opposing the contact face, the retention face defining a retention plane orientated at a compound angle relative to the mounting surface, the engagement of the contact face of the third coupling member and the retention face of the proximal retaining portion being configured to develop a first force parallel to the mounting surface and a second force orthogonal to the mounting surface.

7. The cartridge assembly of claim 6, wherein the third coupling member further includes:
a hard-stop positioned to engage the proximal retaining portion and limit a longitudinal movement of the cartridge chassis in a proximal direction.

8. The cartridge assembly of claim 6, wherein:
the living hinge of the third coupling member is a third living hinge;
the contact face of the third coupling member is a third contact face; and
the proximal perimeter portion of the cartridge chassis includes a fourth coupling member of the plurality of coupling members, the fourth coupling member being positioned opposite the third coupling member about a longitudinal axis of the cartridge chassis, the fourth coupling member including:
a fourth living hinge defined by the proximal perimeter portion and having a neutral position when the cartridge chassis is separated from the delivery device and a compressed position when received by the coupling interface, the fourth living hinge being oriented to exert a force on the delivery device when received by the coupling interface, and
a fourth contact face engaged between the mounting surface and the proximal retaining portion of the coupling interface of the delivery device.

9. The cartridge assembly of claim 2, wherein the cartridge chassis includes:
a plunger guide structure in axial alignment with the container clip, the plunger guide structure defining a receiving volume having a radius that is less than a radius of a receiving volume defined by the container clip.

10. The cartridge assembly of claim 9, wherein:
the container clip defines a nominal positioning indicator corresponding to a nominal position of a distal end of the elastomeric member when the container is secured by the container clip.

11. The cartridge assembly of claim 9, wherein:
the cartridge chassis defines a recess positioned between and aligned with the plunger guide structure and the container clip, the recess having a long edge oriented orthogonal to a longitudinal axis of the cartridge chassis, the recess being sized to receive a portion of a flange of the container secured by the cartridge chassis.

12. The cartridge assembly of claim 11, wherein:
the recess is sized such that a longitudinal axis of the container body is parallel to a mounting surface of the coupling interface when the portion of the flange of the container is within the recess and when the cartridge chassis is received by the coupling interface of the delivery device.

13. The cartridge assembly of claim 11, wherein the cartridge chassis further includes:
a stop protrusion positioned between the recess and the container clip to engage the flange of the container and preclude a movement of the container housing in a distal direction.

14. The cartridge assembly of claim 2, wherein the cartridge chassis further includes:
a sensor interface feature configured to operably engage with a positional sensor of the delivery device, the sensor interface feature being sized to preclude the engagement of the positional sensor for a longitudinal position of the cartridge chassis that deviates from a nominal longitudinal position.

15. The cartridge assembly of claim 2, wherein the cartridge chassis includes:
a manual engagement structure positioned to facilitate removal of the cartridge chassis from the delivery device.

16. The cartridge assembly of claim 2, wherein:
the container is fixed in a first coordinate system defined by the cartridge chassis; and
the cartridge chassis is fixed in a second coordinate system defined by the delivery device.

17. The cartridge assembly of claim 2, wherein the container is a first container containing a first component of the composition, the container clip is a first container clip, the cartridge assembly further includes:
a second container containing a second component of the composition, the second container being at least partially surrounded by a second container clip of the cartridge chassis such that a longitudinal axis of the second container is parallel to a longitudinal axis of the first container.

18. The cartridge assembly of claim 17, further including:
a connector coupled to a distal end of the first container and to a distal end of the second container.

19. The cartridge assembly of claim 17, further including:
a plunger link coupled to a plunger of the first container and a plunger of the second container such that the plunger of the first container and the plunger of the second container move distally in unison in response to an input from the delivery device.

20-38. (canceled)
